SEQUENCE LISTING (5 sequences)

SEQ ID NO: 1 - DNA, 200 bp, synthetic construct
  source 1..200: mol_type = other DNA
tcccaatcgtagcttcctgtggtgtatctgaattctttctccctttaatccaggtgagtgaagagctgattcgggtagccatcctctggcatgagatgtggcatgaaggattggaagaagcaactcgcctgtacttcggagagaggaacgtgaaaggcatgtttgaggtgctggagcccctgcatgctatgatggaacgg

SEQ ID NO: 2 - DNA, 20 bp, synthetic construct
  source 1..20: mol_type = other DNA
acaagcgagatgcctcttcc

SEQ ID NO: 3 - DNA, 20 bp, synthetic construct
  source 1..20: mol_type = other DNA
gaggcatctcgcttgtactt

SEQ ID NO: 4 - DNA, 20 bp, synthetic construct
  source 1..20: mol_type = other DNA
aggcatctcgcttgtacttt

SEQ ID NO: 5 - DNA, 20 bp, synthetic construct
  source 1..20: mol_type = other DNA
ggcatctcgcttgtactttg

(19) United States
(12) Patent Application Publication
(10) Pub. No.: US 2025/0325528 A1
(43) Pub. Date: Oct. 23, 2025

(54) THERAPEUTIC ROLE OF mTORC1 INHIBITION OF NON-TUMOR CELLS

(71) Applicant: THE BOARD OF REGENTS OF THE UNIVERSITY OF TEXAS SYSTEM, Austin, TX (US)

(72) Inventors: James Brugarolas, Irving, TX (US); Juan Yang, Dallas, TX (US); Ramesh Butti, Dallas, TX (US); Shannon Cohn, Dallas, TX (US)

(73) Assignee: THE BOARD OF REGENTS OF THE UNIVERSITY OF TEXAS SYSTEM, Austin, TX (US)

(21) Appl. No.: 19/083,289

(22) Filed: Mar. 18, 2025

Related U.S. Application Data
(60) Provisional application No. 63/566,660, filed on Mar. 18, 2024.

(51) Int. Cl. A61K 31/439; A61K 31/5377; A61P 35/00; C12Q 1/6886; G01N 33/574

(52) U.S. Cl. CPC A61K 31/439; A61K 31/5377; A61P 35/00; C12Q 1/6886; G01N 33/57407; C12Q 2600/106; C12Q 2600/156; G01N 2440/14; G01N 2500/04; G01N 2500/10

(57) ABSTRACT

Provided herein are methods for determining whether a subject is suitable for mTORC1 inhibitor treatments, as well as methods for determining whether an ongoing mTORC1 inhibitor treatment should continue.

### CROSS-REFERENCE TO RELATED APPLICATION

This application claims priority to U.S. Provisional Application Ser. No. 63/566,660 filed on Mar. 18, 2024, and titled “THERAPEUTIC ROLE OF mTORC1 INHIBITION OF NON-TUMOR CELLS,” the disclosure of which is hereby incorporated by reference in its entirety.

### ACKNOWLEDGEMENT OF GOVERNMENT SUPPORT

This invention was made with government support under grant no. CA196516 awarded by the National Institutes of Health. The government has certain rights in the invention.

### SEQUENCE LISTING

The instant application contains a Sequence Listing which has been submitted electronically in XML format and is hereby incorporated by reference in its entirety for all purposes. The XML copy, created on Mar. 17, 2025, is referred to as UTSD.P3882US_SequenceListing.xml and is 5,641 bytes in size.

### BACKGROUND

### 1. Field

The present disclosure relates generally to mTORC1 inhibitors and more specifically to methods for identifying mTORC1 inhibitor resistance and modifying treatment regimens in subjects receiving mTORC1 inhibitor therapy.

### 2. Discussion of Related Art

Renal cell carcinoma (RCC) is one of the 10 most common cancer types. The most common subtype is clear-cell RCC (ccRCC), which accounts for about 75% of cases. Mutation of the von Hippel-Lindau (VHL) tumor suppressor gene initiates ccRCC development. The VHL protein (pVHL) suppresses hypoxia-inducible factor (HIF)1a/2a transcription factors. Abnormal HIF accumulation in ccRCC drives a pseudohypoxic response, leading to VEGF induction and angiogenesis. A second pathway implicated in ccRCC is governed by mammalian (or mechanistic) target of rapamycin complex 1 (mTORC1). mTORC1 promotes cell growth and is frequently activated in ccRCC (as well as non-ccRCC renal cancers). These discoveries laid the foundation for the development of multiple targeted therapies, and today, nearly a dozen drugs inhibiting VEGF/VEGF receptor-2 and mTORC1 are approved by the FDA for advanced RCC treatment.

Two specific mTORC1 inhibitors, temsirolimus and everolimus, both rapamycin analogs (rapalogs), are FDA-approved for the treatment of RCC. Everolimus is also approved in combination with the angiogenesis inhibitor lenvatinib for salvage therapy. Despite their activity, the use of rapalogs and more generally mTORC1 inhibitors is limited due to the acquisition of resistance during treatment. How resistance arises remains unknown, which is particularly striking considering that rapalogs were introduced into the clinic more than a decade ago. Understanding how resistance occurs can also illuminate mechanisms of drug action.

Unlike conventional kinase inhibitors, rapalogs are highly specific allosteric inhibitors of mTORC1. They form a complex with FK506-binding protein 12, FKBP12 (or related proteins), and as a complex, bind to the FKBP12 rapamycin-binding (FRB) domain of mTOR. Inhibition of mTORC1 blocks phosphorylation of downstream targets, in particular, the S6 kinase (S6K)/ribosomal S6 effector pathway, and S6 phosphorylation is commonly used as a readout. Mutations in the FRB domain are known to confer resistance to rapalogs. However, such mutations have not been identified in RCC.

### SUMMARY

The present disclosure is based on the seminal discovery that mTOR pathway inhibition of tumor microenvironment cells is required for mTOR inhibitor activity against cancer cells. In particular, it was determined herein that mTORC1 inhibition of tumor microenvironment cells is required for mTORC1 inhibitor activity against cancer cells. While it was previously hypothesized that mTOR pathway inhibitor resistance was primarily conferred by acquired resistance in cancer cells, it was surprisingly determined herein that mTOR pathway inhibitor resistance in nearby cells, such as stromal cells present in the microenvironment of cancer cells, can diminish the efficacy of treatment of mTOR pathway inhibitors. These findings support a model in which mTOR (e.g., mTORC1) inhibition is required in both cancer cells and nearby non-cancer cells for effective treatment. Furthermore, these findings support a model in which acquired mTOR inhibitor resistance in tumor microenvironment cells can render a subject unsuitable for a particular mTOR inhibitor treatment.

Leveraging this discovery, in one aspect, the present disclosure provides a method of identifying an mTORC1 inhibitor for disease treatment that includes: a) contacting a cell derived from a tumor microenvironment with the mTOR inhibitor and b) detecting mTOR inhibition in the cell, thereby identifying the mTOR inhibitor for cancer treatment. Accordingly, in another aspect, the present disclosure provides a method of identifying an mTORC1 inhibitor for disease treatment that includes: a) contacting a cell derived from a tumor microenvironment with the mTORC1 inhibitor and b) detecting mTORC1 inhibition in the cell, thereby identifying the mTORC1 inhibitor for cancer treatment.

In one embodiment, the detecting is in vitro. In one embodiment, the detecting includes measuring a level of mTORC1 phosphorylation, S6 kinase, ribosomal S6, or 4EBP, or a combination thereof. In another embodiment, the detecting includes measuring a level of other phosphorylated substrates. In an additional embodiment, the detecting includes measuring a decrease in ATP hydrolysis by mTORC1 isolated from the cell. In a further embodiment, the detecting includes measuring a level of binding of the mTORC1 inhibitor to the mTORC1 isolated from the cell or FKBP12 isolated from the cell.

In another embodiment, the method further includes measuring proliferation of a tumor cell in the tumor microenvironment in the presence of the mTORC1 inhibitor.

In one embodiment, the disease is cancer. In another embodiment, the cancer is renal cancer. In an additional embodiment, the renal cancer is clear-cell renal cell carcinoma. In some embodiments, the renal cancer is non-clear-cell renal cell carcinoma (nccRCC). In a further embodiment, the cell is an immune cell, stromal cell, or fibroblast. In one aspect, the cell is a fibroblast.

In one embodiment, the method further includes detecting mTORC1 inhibition in a cancer cell. In a particular embodiment, the cancer cell is derived from the tumor microenvironment.

In a further embodiment, the method further includes determining that mTORC1 is inhibited in the cell for at least about 3 days, at least about 5 days, at least about 8 days, at least about 12 days, at least about 15 days, at least about 20 days, at least about 25 days, at least about 30 days, at least about 50 days, at least about 80 days, at least about 120, at least about 150 days, at least about 200 days, at least about 250 days, at least about 300 days, or at least about 500 days. In additional embodiments, the mTORC1 inhibitor is contacted to the cell between about 4 times per day and once every 15 days, between about 4 times per day and once per day, between about once every 1 to 5 days, between about once every 2 to 10 days, between about once every 3 to 15 days, or between about once every 5 to 15 days.

In another aspect, the present disclosure provides a method of selecting a subject for treatment of a disease with an mTORC1 inhibitor including: a) contacting the mTORC1 inhibitor to a cell derived from a tumor microenvironment of the subject or mTORC1 isolated from the cell; b) detecting inhibition of mTORC1 in the cell or the mTORC1 isolated from the cell; and c) selecting the subject for treatment of the disease with the mTORC1 inhibitor. In further aspects, the present disclosure provides a method of selecting a subject for treatment of a disease with an mTORC1 inhibitor including: a) contacting the mTORC1 inhibitor to a cell derived from a tumor microenvironment of the subject or mTORC1 isolated from the cell; b) detecting inhibition of mTORC1 in the cell or the mTORC1 isolated from the cell; and c) selecting the subject for treatment of the disease with the mTORC1 inhibitor.

In some embodiments, the method includes contacting the mTORC1 inhibitor to the cell and detecting the inhibition of the mTORC1 in the cell. In some embodiments, the method includes contacting the mTORC1 inhibitor to the mTORC1 isolated from the cell and detecting the inhibition of the mTORC1 isolated from the cell. In some embodiments, the detecting is in vitro. In some embodiments, the detecting includes measuring a level of phosphorylation of the mTORC1, S6 kinase, ribosomal S6, or 4EBP, or a combination thereof in the cell. In some embodiments, the detecting includes measuring a level of phosphorylated S6 kinase in the cell. In some embodiments, the detecting includes measuring a decrease in ATP hydrolysis by the mTORC1 isolated from the cell. In some embodiments, the detecting includes measuring a level of binding of the mTORC1 inhibitor to the mTORC1 isolated from the cell or FKBP12 isolated from the cell.

In some embodiments, the detecting includes precipitating the mTORC1 isolated from the cell and measuring activity of the mTORC1 in the presence of a protein substrate and ATP.

In some embodiments, the protein substrate is S6 kinase or 4EBP. In some embodiments, the cell is derived from a tissue or microenvironment that contains a diseased cell.

In some embodiments, the disease is cancer. In some embodiments, the cancer is renal cancer. In some embodiments, the renal cancer is clear-cell renal cell carcinoma (ccRCC). In some embodiments, the renal cancer is non-clear-cell renal cell carcinoma (nccRCC).

In some embodiments, the cell is derived from a tumor microenvironment of the cancer. In some embodiments, the cell is an immune cell, stromal cell, or fibroblast.

In some embodiments, the mTORC1 inhibitor is rapamycin, MTI-31, dactolisib, deforolimus, ridaforolimus, temsirolimus, everolimus, zotarolimus, RAD001, CCI-779, KU-0063794, WYE-354, GSK1059615, AZD8055, nitazoxanide, trokinib, palomid 529, PP121, OSI-027, NU7441, omipalisib, WYE-125132, WYE-687, WAY-600, sapanisertib, torin 1, torin 2, zotarolimus, onatasertib, CZ415, PZR620, or an analogue thereof. In some embodiments, the mTORC1 inhibitor is rapamycin or a rapamycin analogue. In some embodiments, the rapamycin analogue is deforolimus, ridaforolimus, temsirolimus, everolimus, zotarolimus, RAD001, or CCI-779.

In some embodiments, the method further includes: d) repeating the detecting inhibition of mTORC1 in the cell or the mTORC1 isolated from the cell subsequently to the selecting the subject. In some embodiments, d) is performed about 3 days to 600 days, about 3 days to 15 days, about 10 days to 50 days, about 20 days to 100 days, about 40 days to 200 days, about 60 days to 300 days, or about 120 days to 600 days after c). In some embodiments, the method further includes continuing treatment with the mTORC1 inhibitor.

In another aspect, the present disclosure provides a method of selecting a subject for treatment of a disease with an mTORC1 inhibitor including: analyzing a cell isolated from a tumor microenvironment of the subject for an mTORC1 inhibitor resistance marker; and selecting the subject for treatment of the disease with the mTORC1 inhibitor.

In a specific aspect, the present disclosure provides a method of selecting a subject for treatment of a disease with an mTORC1 inhibitor including: analyzing a cell isolated from a tumor microenvironment of the subject for a mTORC1 inhibitor resistance marker; and selecting the subject for treatment of the disease with the mTORC1 inhibitor. In a specific aspect, the present disclosure provides a method of selecting a subject for treatment of a disease with an mTORC1 inhibitor including: analyzing a cell isolated from a tumor microenvironment of the subject for a mTORC1 inhibitor resistance marker; and selecting the subject for treatment of the disease with the mTORC1 inhibitor.

In some embodiments, the disease is cancer. In some embodiments, the cancer is renal cancer. In some embodiments, the renal cancer is clear-cell renal cell carcinoma. In some embodiments, the renal cancer is non-clear-cell renal cell carcinoma (nccRCC). In some embodiments, the cell is derived from a tumor microenvironment of the cancer. In some embodiments, the cell is an immune cell, a stromal cell, or a fibroblast. In some embodiments, the stromal cell is a fibroblast. In some embodiments, the cell is a fibroblast.

In some embodiments, the mTORC1 inhibitor is rapamycin, MTI-31, dactolisib, deforolimus, ridaforolimus, temsirolimus, everolimus, zotarolimus, RAD001, CCI-779, KU-0063794, WYE-354, GSK1059615, AZD8055, nitazoxanide, trokinib, palomid 529, PP121, OSI-027, NU7441, omipalisib, WYE-125132, WYE-687, WAY-600, sapanisertib, torin 1, torin 2, zotarolimus, onatasertib, CZ415, PZR620, or an analogue thereof. In some embodiments, the mTORC1 inhibitor is rapamycin or an analogue thereof.

In some embodiments, determining the absence of a resistance marker for the mTORC1 inhibitor includes profiling the subject for a genetic mutation. In some embodiments, the genetic mutation is associated with increased or constitutive mTORC1 activity. In some embodiments, the cell is isolated from a tissue or microenvironment containing a diseased cell. In some embodiments, the cell is isolated from a tumor microenvironment.

In a further aspect, the present disclosure provides a method of selecting a treatment method for treating a subject with a disease in need thereof including: a) contacting the mTORC1 inhibitor to a cell derived from a tumor microenvironment of the subject or mTORC1 isolated from the cell; b) detecting activity of mTORC1 in the cell or the mTORC1 isolated from the cell; and c) administering the mTORC1 inhibitor to the subject if the activity of the mTORC1 in the cell or the mTORC1 isolated from the cell is inhibited.

In some embodiments, the detecting includes measuring a level of phosphorylation of mTORC1, S6 kinase, ribosomal S6, or 4EBP, or a combination thereof in the cell. In some embodiments, the detecting includes measuring a level of phosphorylated S6 kinase in the cell.

In some embodiments, the detecting includes measuring a decrease in ATP hydrolysis by the mTORC1 isolated from the cell. In some embodiments, the detecting includes measuring a level of binding of the mTORC1 inhibitor to the mTORC1 isolated from the cell or FKBP12 isolated from the cell. In some embodiments, the detecting includes precipitating the mTORC1 isolated from the cell and measuring activity of the mTORC1 in the presence of a protein substrate and ATP.

In some embodiments, the protein substrate is S6 kinase or 4EBP. In some embodiments, the cell is derived from a tissue or microenvironment that contains a diseased cell.

In some embodiments, the disease is cancer. In some embodiments, the cancer is renal cancer. In some embodiments, the renal cancer is clear-cell renal cell carcinoma (ccRCC). In some embodiments, the renal cancer is non-clear-cell renal cell carcinoma (nccRCC).

In some embodiments, the cell is derived from a tumor microenvironment of the cancer. In some embodiments, the cell is an immune cell, stromal cell, or fibroblast.

In some embodiments, the mTORC1 inhibitor is rapamycin, MTI-31, dactolisib, deforolimus, ridaforolimus, temsirolimus, everolimus, zotarolimus, RAD001, CCI-779, KU-0063794, WYE-354, GSK1059615, AZD8055, nitazoxanide, trokinib, palomid 529, PP121, OSI-027, NU7441, omipalisib, WYE-125132, WYE-687, WAY-600, sapanisertib, torin 1, torin 2, zotarolimus, onatasertib, CZ415, PZR620, or an analogue thereof. In some embodiments, the mTORC1 inhibitor is rapamycin or a rapamycin analogue. In some embodiments, the rapamycin analogue is deforolimus, ridaforolimus, temsirolimus, everolimus, zotarolimus, RAD001, or CCI-779.

In some embodiments, the method further includes determining that the mTORC1 is still inhibited at least 1 day, at least 2 days, at least 3 days, at least 4 days, at least 5 days, at least 6 days, at least 8 days, at least 10 days, at least 12 days, at least 15 days, at least 20 days, at least 25 days, at least 30 days, at least 40 days, at least 50 days, at least 80 days, at least 120 days, at least 150 days, or at least 200 days after the administering the mTORC1 inhibitor. In some embodiments, the method further includes continuing administration of the mTORC1 inhibitor.

In another aspect, the present disclosure provides a method of selecting a treatment method for treating a subject with a disease in need thereof including: a) analyzing a cell isolated from a tumor microenvironment of the subject for a mTORC1 inhibitor resistance marker; b) selecting the subject for treatment of the disease with the mTORC1 inhibitor.

In some embodiments, the method includes administering the mTOR inhibitor to the subject.

In some embodiments, the mTORC1 inhibitor is rapamycin, MTI-31, dactolisib, deforolimus, ridaforolimus, temsirolimus, everolimus, zotarolimus, RAD001, CCI-779, KU-0063794, WYE-354, GSK1059615, AZD8055, nitazoxanide, trokinib, palomid 529, PP121, OSI-027, NU7441, omipalisib, WYE-125132, WYE-687, WAY-600, sapanisertib, torin 1, torin 2, zotarolimus, onatasertib, CZ415, PZR620, or an analogue thereof. In some embodiments, the mTORC1 inhibitor is rapamycin or an analogue thereof.

A further aspect of the present disclosure provides a method of treating a subject with a disease including: a) administering an mTORC1 inhibitor to the subject for a first time period; b) ceasing administration of the mTORC1 inhibitor for a second time period following the first time period; and c) resuming administration of the mTORC1 inhibitor following the second time period for a third time period.

In some embodiments, the method further includes detecting resistance to the mTORC1 inhibitor in a tumor microenvironment cell from the subject. In some embodiments, resistance to the mTORC1 inhibitor is measured in tumor microenvironment cells from the subject about every 1, 2, 3, 4, 5, 6, 7, 8, 9, 10, 12, 15, 20, 25, 30, 40, 50, or 60 days. In some embodiments, the method further includes ending the first time period upon detecting resistance to the mTORC1 inhibitor resistance in the tumor microenvironment cell. In some embodiments, the second time period is about 2 to 60 days, about 2 to 5 days, about 2 to 10 days, about 2 to 20 days, about 5 to 20 days, about 5 to 40 days, about 10 to 40 days, about 10 to 60 days, about 20 to 60 days, about 30 to 60 days, or about 40 to 60 days. In some embodiments, the subject is selected by a method described herein.

In some embodiments, the mTORC1 inhibitor is rapamycin, MTI-31, dactolisib, deforolimus, ridaforolimus, temsirolimus, everolimus, zotarolimus, RAD001, CCI-779, KU-0063794, WYE-354, GSK1059615, AZD8055, nitazoxanide, trokinib, palomid 529, PP121, OSI-027, NU7441, omipalisib, WYE-125132, WYE-687, WAY-600, sapanisertib, torin 1, torin 2, zotarolimus, onatasertib, CZ415, PZR620, or an analogue thereof. In some embodiments, the mTORC1 inhibitor is rapamycin or an analogue thereof.

In some embodiments, the disease is cancer. In some embodiments, the cancer is renal cancer. In some embodiments, the renal cancer is clear-cell renal cell carcinoma. In some embodiments, the renal cancer is non-clear-cell renal cell carcinoma (nccRCC). In some embodiments, the cell is derived from a tumor microenvironment of the cancer. In some embodiments, the cell is an immune cell, a stromal cell, or a fibroblast. In some embodiments, the stromal cell is a fibroblast. In some embodiments, the cell is a fibroblast.

In a further aspect, the present disclosure provides a method of treating a subject with a disease that includes: a) analyzing a cell derived from a tumor microenvironment from the subject for a mTORC1 inhibitor resistance marker; and b) administering to the subject an mTORC1 inhibitor.

In some embodiments, the mTORC1 inhibitor is rapamycin, MTI-31, dactolisib, deforolimus, ridaforolimus, temsirolimus, everolimus, zotarolimus, RAD001, CCI-779, KU-0063794, WYE-354, GSK1059615, AZD8055, nitazoxanide, trokinib, palomid 529, PP121, OSI-027, NU7441, omipalisib, WYE-125132, WYE-687, WAY-600, sapanisertib, torin 1, torin 2, zotarolimus, onatasertib, CZ415, PZR620, or an analogue thereof. In some embodiments, the mTORC1 inhibitor is rapamycin or an analogue thereof.

In some embodiments, the disease is cancer. In some embodiments, the cancer is renal cancer. In some embodiments, the renal cancer is clear-cell renal cell carcinoma. In some embodiments, the renal cancer is non-clear-cell renal cell carcinoma (nccRCC). In some embodiments, the cell is derived from a tumor microenvironment of the cancer. In some embodiments, the cell is an immune cell, a stromal cell, or a fibroblast. In some embodiments, the stromal cell is a fibroblast. In some embodiments, the cell is a fibroblast.

In another aspect, the present disclosure provides a method of determining the role of the tumor microenvironment in response to a therapy comprising: i) transplanting a first tumor or tumor cell into a first immunocompromised mammal comprising a mutation and a second immunocompromised mammal that does not comprise the mutation; ii) administering the therapy to the first and second immunocompromised mammals; and iii) comparing a response of the tumor or tumor cell in the first and second immunocompromised mammals; thereby determining the role of the tumor microenvironment in response to the therapy.

In some embodiments, the first and second immunocompromised mammals are mice.

In some embodiments, the first and second immunocompromised mammals are NOD or SCID mice.

In some embodiments, the mutation is an mTOR mutation. In some embodiments, the therapy comprises an mTOR inhibitor.

In further embodiments, the mutation confers resistance to the therapy. In particular embodiments, the mutation is in a putative target of the therapy. In specific embodiments: i) the mutation is an EGFR (epidermal growth factor receptor) mutation and the therapy comprises an EGFR inhibitor; ii) the mutation is an Abl kinase mutation and the therapy comprises an Abl kinase inhibitor; or iii) the mutation is a Ret mutation (i.e., a RET proto-oncogene mutation) and the therapy comprises a Ret inhibitor.

In one embodiment, a method is provided for identifying a subject for treatment with an mTOR1 inhibitor, the method comprising: a) i) contacting the mTORC1 inhibitor to a cell derived from a tumor microenvironment of the subject or mTORC1 isolated from the cell and detecting inhibition of mTORC1 in the cell or the mTORC1 isolated from the cell; and/or ii) analyzing a cell derived from a tumor microenvironment from the subject for an mTORC1 inhibitor resistance marker; and b) identifying the subject for treatment with the mTORC1 inhibitor if mTORC1 inhibition is detected and/or if the mTORC1 inhibitor resistance marker is not detected.

In various embodiments, detecting mTORC1 inhibition comprises measuring a level of phosphorylation of the mTORC1, S6 kinase, ribosomal S6, or 4EBP, or a combination thereof.

In various embodiments, the method comprises detecting inhibition of mTORC1 isolated from the cell. For example, in some aspects, step a) i) comprises: 1) measuring a decrease in ATP hydrolysis by the mTORC1 isolated from the cell; and/or 2) measuring a level of binding of the mTORC1 inhibitor to the mTORC1 isolated from the cell or FKBP12 isolated from the cell; and/or 3) precipitating the mTORC1 isolated from the cell and measuring activity of the mTORC1 in the presence of a protein substrate (e.g., S6 kinase or 4EBP) and ATP.

In some embodiments, analyzing the cell for an mTORC1 inhibitor resistance marker comprises profiling the subject for a genetic mutation. In some aspects, the genetic mutation is associated with increased or constitutive mTORC1 activity.

In any of the foregoing or related embodiments, the cell derived from a tumor microenvironment of the subject is an immune cell, stromal cell, or fibroblast.

In further embodiments, the methods provided herein may further comprise a step d) repeating detecting inhibition of mTORC1 in the cell or the mTORC1 isolated from the cell and/or repeating analyzing a cell derived from a tumor microenvironment from the subject for an absence of the mTORC1 inhibitor resistance marker and e) continuing treatment with the mTORC1 inhibitor if mTORC1 inhibition is detected and/or the mTORC1 inhibitor resistance marker is absent. In some aspects, d) is performed about 3 days to 600 days, about 3 days to 15 days, about 10 days to 50 days, about 20 days to 100 days, about 40 days to 200 days, about 60 days to 300 days, or about 120 days to 600 days after c).

In any of these embodiments, the detecting (e.g., detecting mTORC1 inhibition and/or an mTORC1 inhibitor resistor marker) may be performed in vitro.

In any of these embodiments, the cancer is renal cancer. In some embodiments, the renal cancer is clear-cell renal cell carcinoma or non-clear-cell renal cell carcinoma.

In any of these embodiments, the mTORC1 inhibitor is rapamycin, MTI-31, dactolisib, deforolimus, ridaforolimus, temsirolimus, everolimus, zotarolimus, RAD001, CCI-779, KU-0063794, WYE-354, GSK1059615, AZD8055, nitazoxanide, trokinib, palomid 529, PP121, OSI-027, NU7441, omipalisib, WYE-125132, WYE-687, WAY-600, sapanisertib, torin 1, torin 2, zotarolimus, onatasertib, CZ415, PZR620, or an analogue thereof. In some embodiments, the mTORC1 inhibitor is rapamycin, deforolimus, ridaforolimus, temsirolimus, everolimus, zotarolimus, RAD001, CCI-779 or an analogue thereof.

In another embodiment, a method of treating a subject with cancer is provided, the method comprising a) administering an mTORC1 inhibitor to the subject for a first time period; b) ceasing administration of the mTORC1 inhibitor for a second time period following the first time period if the disease has been determined to be resistant to the mTORC1 inhibitor; and c) resuming administration of the mTORC1 inhibitor following the second time period for a third time period.

In various embodiments, the cancer has been determined to be resistant to the mTORC1 inhibitor when a cell derived from the tumor microenvironment is resistant to mTORC1 inhibition and/or has an mTORC1 inhibitor resistance marker. Accordingly, in various embodiments, the method further comprises measuring the resistance to the mTORC1 inhibitor and/or detecting an mTORC1 inhibitor resistance marker in a cell derived from the tumor microenvironment from the subject about every 1, 2, 3, 4, 5, 6, 7, 8, 9, 10, 12, 15, 20, 25, 30, 40, 50, or 60 days.

In some embodiments, the second time period is about 2 to 60 days, about 2 to 5 days, about 2 to 10 days, about 2 to 20 days, about 5 to 20 days, about 5 to 40 days, about 10 to 40 days, about 10 to 60 days, about 20 to 60 days, about 30 to 60 days, or about 40 to 60 days.

In another embodiment, a method of treating a subject with an mTORC1 inhibitor is provided, the method comprising administering the mTORC1 inhibitor to the subject, wherein the subject has been identified for treatment with the mTORC1 inhibitor according to any embodiment herein.

In another embodiment, a method of determining the role of the tumor microenvironment in response to a therapy is provided, the method comprising: i) transplanting a first tumor or tumor cell into a first immunocompromised mammal comprising a mutation and a second immunocompromised mammal that does not comprise the mutation; ii) administering the therapy to the first and second immunocompromised mammals; and iii) comparing a response of the tumor or tumor cell in the first and second immunocompromised mammals; thereby determining the role of the tumor microenvironment in response to the therapy, wherein the mutation is an mTOR1 mutation and the therapy comprises an mTOR1 inhibitor; the mutation is an EGFR (epidermal growth factor receptor) mutation and the therapy comprises an EGFR inhibitor; the mutation is an Abl kinase mutation and the therapy comprises an Abl kinase inhibitor; or the mutation is a Ret mutation (i.e., a RET proto-oncogene mutation) and the therapy comprises a Ret inhibitor.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E are a series of graphs, images and RPPA analyses that demonstrate prolonged rapamycin treatment results in acquired resistance despite persistent mTORC1 inhibition in tumor cells. FIG. 1A is a series of growth curves of subcutaneously implanted tumorgrafts (TGs) (XP144, XP164 and XP490) in NOD/SCID mice treated with vehicle (Ve) or rapamycin (Ra). FIG. 1B is a set of representative phospho-S6 immunohistochemistry images of TGs from vehicle-treated (Ve) mice or mice treated with rapamycin collected while TGs were sensitive (˜day 28) (Ra^{sensitive}) and after development of resistance (Ra^{Resistant}). FIG. 1C displays the quantitation of the results shown in FIG. 1B. FIG. 1D is a series of Western blot analyses of mTORC1 pathway status of TGs from vehicle-treated mice or mice treated with rapamycin collected while TGs were sensitive (Ra^{sensitive}) and after development of resistance (Ra^{Resistant}) For each condition, 3 different TGs are shown per TG line. P-S6, phospho-S6 Ser240/244. FIG. 1E summarizes RPPA analyses of (phospho)protein levels of rapamycin-sensitive and rapamycin-resistant TGs compared with vehicle-treated tumors for each TG line. Heatmap of selected (phospho)protein levels of TGs from vehicle-treated mice or mice treated with rapamycin collected while TGs were sensitive (Ra^{sensitive}) and after development of resistance (Ra^{Resistant})

FIGS. 2A-2E are a series of growth curves, Western blot analyses and immunofluorescence images that show that rapamycin resistance is reversible and lost with both subsequent transplantation and in cell culture. FIG. 2A is a series of TG growth curves following transplantation of rapamycin-resistant tumors to a new cohort of mice. FIG. 2B is a series of representative TG growth curve of rapamycin-resistant tumor transplanted into a third mouse cohort. FIG. 2C is a series of representative Western blot analyses of sequentially transplanted tumors in second and third cohorts for S6 phosphorylated (P-S6) at Ser240/244. FIG. 2D is a series of images from immunofluorescent staining of TG-harvested primary cells with RCC markers, vimentin (left column) and PAX8 (middle column). FIG. 2E is a series of primary cell growth curves from vehicle-treated or Ra^{Resistant} TGs in mice cultured with vehicle (Ve) or 50 nM rapamycin (Ra).

FIGS. 3A-3D are a series of images and image analyses that show rapamycin resistance is accompanied by mTORC1 reactivation in the tumor microenvironment. FIGS. 3A and 3B are a series of immunofluorescence analyses of vehicle, Ra^{sensitive} and Ra^{Resistant} TGs for p-S6 and Pax8. White arrows indicate pS6-stained stromal cells (PAX8−) in Ra^{Resistant} TGs. FIGS. 3C and 3D are a set of plots of quantitation of p-S6 positive cells according to PAX8 status. Bar graph represents percent cell populations with p-S6 staining in PAX8+ (tumor) and PAX8− (stromal) cells in different conditions.

FIGS. 4A-4I are a cartoon illustration of a mouse model as well as images of Western blots and plots of DNA analyses that collectively demonstrate the successful generation of an mTOR-S2035T NOD/SCID mouse strain resistant to rapamycin. FIG. 4A is a cartoon illustrating implanted tumor growth in rapamycin-resistant and non-rapamycin resistant mice during rapamycin and vehicle treatments. FIG. 4B is a series of Western blot analyses of HeLa cells transfected with empty vector (EV), wild-type mTOR (mTOR-WT) and mTOR S2035T (mTOR-ST) expression vectors and treated (or not) with rapamycin (50 nM, 45 min). FIG. 4C is an image of DNA electrophoresis of Surveyor test results from sgRNAs targeting MTOR (or an empty vector (EV) control) in NIH/3T3 cells. Arrows indicate Surveyor nuclease S digestion fragments. FIG. 4D is an image of gel electrophoresis analyses showing selective DNA amplification of NIH/3T3 cells transfected with sgRNA+template using a primer containing the EcoR1 consensus sequence in the ssODN template. FIG. 4E is a plot summarizing Sanger sequencing results that illustrate ssODN template utilization with mutation incorporation in NIH/3T3 cells. FIG. 4F is a set of genotypic analyses of DNA from the pups generated from the injection of sgRNAs (sgRNA1 and sgRNA4), ssODN (containing the S2035T mutation), and recombinant SpCas9 into fertilized nuclei of NOD/SCID mouse zygotes, by restriction fragment length polymorphism following PCR amplification and EcoR1 digestion for the introduced restriction site. Two positive founders were obtained (#1 and #2). Arrows indicate EcoRI digestion fragments that show the presence of the mutation. FIG. 4G provides results from bidirectional Sanger sequencing from founder mouse DNA, confirming the introduction of resistance mutation, silent mutations, and the EcoRI site. FIG. 4H is a series of Western blot analyses of MEFs derived from wild-type or NOD/SCID^{mTOR-ST}mutant mice treated or untreated with rapamycin (Ra). FIG. 4I is a set of Western blot analyses of mouse kidneys from either wild-type or NOD/SCID^{mTOR-ST} mutant 5-week-old mice treated with rapamycin or vehicle (Ve) and collected 2 hours after the last dose.

FIGS. 5A-5C are a series of plots, immunofluorescence images and Western blot analyses that show that mTORC1 inhibition of non-tumor cells is required for rapamycin anti-tumor activity. FIG. 5A is a series of TG growth curves in wild-type (NOD/SCID^{mTOR-WT}) and mTOR S2035T heterozygous NOD/SCID^{mTOR-ST} (as well as homozygous NOD/SCID^{mTOR-ST/ST}) mice treated with rapamycin (Ra) or vehicle (Ve). FIG. 5B is a set of immunofluorescence images and plots summarizing P6 and PAX8 expression of TGs in mTOR S2035T mice (XP144, NOD/SCID^{mTOR-ST}; XP490, NOD/SCID^{mTOR-ST/ST}) treated with rapamycin (Ra) or vehicle (Ve). Bar graph represents percent cell populations with p-S6 staining in PAX8+ (tumor) and PAX8− (stromal) cells in vehicle/rapamycin treatment groups in an mTOR-ST background. FIG. 5C is a set of Western blot analyses of XP144, XP164 and XP490 TGs in wild-type and NOD/SCID^{mTOR-ST} mice treated with vehicle or after acquisition of resistance to rapamycin. p-S6 and S6 were from gels run in parallel with the same extracts. P-S6, phospho-S6 Ser240/244.

FIGS. 6A-6C are a set of immunofluorescence images and plots showing mTORC1 activation in mTOR-ST myofibroblasts despite rapamycin. FIG. 6A is a set of immunofluorescence analyses (α-SMA [myofibroblasts] and p-S6) of XP144 TGs (NOD/SCID^{mTOR-ST}) as well as XP164 and XP490 TGs (NOD/SCID^{mTOR-ST/ST}) mice treated with rapamycin (Ra) or vehicle (Ve). FIG. 6B is a set of plots summarizing the distribution of p-S6 signal by FACS in tumor cells (CAIX-positive) and myofibroblasts (α-SMA-positive) from TGs implanted into wild-type and NOD/SCID^{mTOR-ST}mice and treated with vehicle or rapamycin compared to a negative control (NC, without phospho-S6 antibody). P-S6, phospho-S6 Ser240/244. FIG. 6C is a set of plots that quantitate the results shown in FIG. 6B.

FIGS. 7A-7C shows the loss of TG resistance following transplantation from NOD/SCIDmTOR-ST into NOD/SCID wild-type mice. FIGS. 7A-7C are a set of TG growth curves of rapamycin-resistant tumors transplanted from NOD/SCID^{mTOR-ST} mice into a new cohort of wild-type mice and treated with vehicle (Ve) or rapamycin (Ra). FIG. 7A is a tumorgraft growth plot for XP144 TGs transplanted subcutaneously into mice. FIG. 7B is a tumorgraft growth plot for XP164 TGs transplanted subcutaneously into mice. FIG. 7C is a tumorgraft growth plot for XP490 TGs transplanted subcutaneously into mice.

FIGS. 8A-8B are a series of plots, and Western blot analyses showing rapalog-resistant fibroblasts dampen rapalog anti-tumor activity in co-culture experiments. FIG. 8A is a set of plots of quantification experiments of primary cells from XP144, XP164 and XP490 TGs co-cultured with wild-type or NOD/SCID^{mTOR-ST}fibroblasts and treated with vehicle or 50 nM rapamycin for 3 days. The error bars indicate SEM. FIG. 8B is a series of Western blot analyses of the co-cultured primary tumor cells or fibroblasts treated with vehicle or rapamycin. P-S6, phospho-S6 Ser240/244.

FIG. 8C is a graphic depiction of a model of rapalog action and resistance. Failure to inhibit mTORC1 in the tumor microenvironment of NOD/SCID^{mTOR-ST} mice induces resistance, enabling tumor growth despite persistent mTORC1 inhibition in tumor cells.

FIG. 9 is a graphic depiction of unsupervised hierarchical clustering of proteome analyses. Unsupervised hierarchical clustering analyses of RPPA samples of 279 (phospho)proteins in vehicle-treated (Ve), rapamycin-sensitive (Ra^{sensitive}) and rapamycin-resistant (Ra^{Resistant}) tumors (n>3 per condition) using HeLa cells as controls.

FIGS. 10A-10H are a series of plots showing persistent mTORC1 activity in mTOR-ST fibroblasts from TG-bearing rapamycin-treated mice. FIG. 10A is a series of plots that show the distribution of phospho-S6 signal by FACS in tumor (CAIX-positive) cells from XP144 TGs in wild-type and NOD/SCID^{mTOR-ST} mice treated with vehicle (Ve) or rapamycin (Ra) compared to a negative control (NC) with no phospho-S6 antibody. FIG. 10B is a set of plots that summarize the results shown in FIG. 10A. FIG. 10C is a set of plots that show the distribution of phospho-S6 signal by FACS in fibroblasts (α-SMA-positive) from XP144 TGs in wild-type and NOD/SCID^{mTOR-ST} mice treated with vehicle or rapamycin compared to a negative control (NC) with no phospho-S6 antibody. FIG. 10D is a set of plots that summarize the results shown in FIG. 10C. FIG. 10E is a set of plots showing the distribution of phospho-S6 signal by FACS in tumor (CAIX-positive) cells from XP164 TGs in wild-type and NOD/SCID^{mTOR-ST} mice treated with vehicle or rapamycin compared to a negative control (NC) with no phospho-S6 antibody. FIG. 10F is a set of plots that summarize the results shown in FIG. 10E. FIG. 10G is a set of plots that summarize the distribution of phospho-S6 signal by FACS in fibroblasts (SMA-positive) from XP164 TGs in wild-type and NOD/SCID^{mTOR-ST} mice treated with vehicle or rapamycin compared to a negative control (NC) with no phospho-S6 antibody. FIG. 10H is a set of plots that summarize the results shown in FIG. 10G.

FIGS. 11A-11F are series of microscopy images and plots showing restoration of tumor cell proliferation after the acquisition of resistance and in rapamycin resistant hosts (mTOR-ST), assessed by Ki67 staining. FIG. 11A is Ki67 staining of XP144 TGs in wild-type mice treated with either vehicle or rapamycin (Ra^{sensitive}) and following the acquisition of resistance (Ra^{Resistant}) as well as in NOD/SCID^{mTOR-ST} mice, treated with vehicle (Ve) or rapamycin (Ra). FIG. 11B is a plot showing the quantification of FIG. 11A data. FIG. 11C is Ki67 staining of XP164 TGs in wild-type mice treated with either vehicle or rapamycin (Ra^{sensitive}) and following the acquisition of resistance (Ra^{Resistant}) as well as in NOD/SCID^{mTOR-ST} mice, treated with vehicle (Ve) or rapamycin (Ra). FIG. 11D is a plot showing the quantification of FIG. 11C data. FIG. 11E is Ki67 staining of XP490 TGs in wild-type mice treated with either vehicle or rapamycin (Ra^{sensitive}) and following the acquisition of resistance (Ra^{Resistant}) as well as in NOD/SCID^{mTOR-ST} mice, treated with vehicle (Ve) or rapamycin (Ra). FIG. 11F is a plot showing the quantification of FIG. 11E data.

### DETAILED DESCRIPTION

### I. Terminology

Before the present compositions and methods are described, it is to be understood that this disclosure is not limited to particular compositions, methods, and experimental conditions described herein, as such compositions, methods, and conditions may vary. It is also to be understood that the terminology used herein is for purposes of describing particular embodiments only, and is not intended to be limiting, since the scope of the present disclosure will be limited only in the appended claims.

As used in this specification and the appended claims, the singular forms “a,” “an,” and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, the term “and/or” includes any and all combinations of one or more of the associated listed items.

As used herein, the term “about” in association with a numerical value is meant to include any additional numerical value reasonably close to the numerical value indicated. For example, and based on the context, the value can vary up or down by 5-10%. For example, for a value of about 100, means 90 to 110 (or any value between 90 and 110).

As used herein and in the claims, the terms “comprising,” “containing,” and “including” are inclusive, open-ended and do not exclude additional unrecited elements, compositional components or method steps. Accordingly, the terms “comprising” and “including” encompass the comparably more restrictive terms “consisting of” and “consisting essentially of.”

As used herein, unless otherwise stated, the terms “cell derived from a tumor microenvironment” and “tumor microenvironment cell” specifically denote non-cancerous cells derived from a tumor microenvironment that contains a cancerous cell.

As used herein, unless otherwise stated, the term “cancer” refers to diseases characterized by the rapid and uncontrolled cell growth, and encompasses all types of cancers, neoplasms, malignantly transformed cells, and tumors. Examples of cancers suitable for the methods disclosed herein include, but are not limited to, brain cancer, breast cancer, cervical cancer, colon cancer, endocrine cancer, liver cancer, renal cancer, lung cancer, prostate cancer, thyroid cancer, melanoma, mesothelioma, ovarian cancer, sarcoma, rhabdomyosarcoma, primary thrombocytosis, testicular cancer, Hodgkin's Disease, lymphoma, esophageal cancer, skin cancer, pancreatic cancer, stomach cancer, medulloblastoma, colorectal cancer, pancreatic cancer, multiple myeloma, neuroblastoma, glioma, glioblastoma multiforme, ovarian cancer, and colorectal cancer. The term “cancer” also includes non-cancerous (benign) tumors that grow in the brain and several areas of the body, including the spinal cord, nerves, eyes, lung, heart, kidneys, and skin.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, it will be understood that modifications and variations are encompassed within the spirit and scope of the instant disclosure. The preferred methods and materials are now described.

### II. Overview

The present disclosure is based on the seminal discovery that effective mTORC1 inhibition in cancer cells requires mTORC1 to also be inhibited in nearby cells (e.g., cells within a tumor microenvironment). The term “tumor microenvironment” as used herein can denote the cells, biomacromolecular and physiological structures (e.g., tumoral connective tissues including extracellular matrix and vascularization), and environment (low pH extracellular fluid) surrounding tumor cells. Tumors typically exhibit close interrelationships with their microenvironments and participate in bidirectional signaling with non-cancerous cells that influences tumor growth, development, and immune escape. Tumor microenvironments also typically contain chemical profiles that are distinct from those of surrounding tissues, including diminished pH and oxygen concentration.

mTORC1 is a protein complex that includes the protein kinase mTOR associated with auxiliary proteins mLST8, RAPTOR, PRAS40, and DEPTOR. mTORC1 activates signals within the mTOR signaling pathway, and actively regulates a diverse array of processes including cell growth, autophagy, metabolism, lipid synthesis, protein synthesis, anabolism, and nutrient sensing. Aberrant mTORC1 signaling can manifest in a range of conditions, including neurodegenerative disorders such as Alzheimer's disease, diabetes, and numerous physiological syndromes and cancers.

While many of these conditions (and in particular many cancers) may not be associated with mTOR mutations, mTOR inhibitor resistance is commonly acquired during mTOR inhibitor treatment. mTOR signaling is regulated by a large number of regulatory proteins. For example, mTORC1 signaling is regulated by a large number of regulatory proteins, including AMPK, TSC, and Rheb. mTOR, and in particular in mTORC1, regulates numerous cellular processes, and is therefore an important target for a variety of conditions and pathologies. mTORC1 is aberrantly activated in a number of cancers, including renal cell carcinoma (RCC), rendering mTORC1 inhibitors useful in the treatment of numerous cancers. As for numerous targeted therapies, the clinical utility of mTORC1 inhibitors is often limited by the drug resistance developed during treatment. While drug resistance often results from mutations in their targets, a number of cancers, including renal cell carcinoma (RCC), rarely exhibit mTOR mutations. Nonetheless, it was shown herein that prolonged exposure of cancer tumorgrafts (TGs) to mTORC1 inhibitors led to mTORC1 inhibitor resistance. Unexpectedly, however, it was also shown that explants from resistant tumors become sensitive both in culture and in subsequent transplants, and that resistance develops despite persistent mTORC1 inhibition in tumor cells. Conversely, mTORC1 reactivation was observed in the tumor microenvironment (TME). These results collectively support a model in which effective mTORC1 inhibitor treatment requires mTORC1 inhibition of diseased cells and bystander cells within the diseased cells' microenvironments. Following from this model, a subject may develop a resistance to an mTORC1 inhibitor that renders mTORC1 inhibitor therapy ineffective, even in cases in which the diseased cells remain responsive to the mTORC1 inhibitor.

The analyses disclosed herein sought to address a longstanding unanswered question of how RCC acquires resistance to rapalogs. The results showed that the status of mTORC1 in non-tumor cells plays a critical role in the response and resistance to rapalogs.

To study acquired resistance, this process was modeled in TGs, where it was observed that prolonged treatment with rapamycin (at exposures matching human exposures) was associated with the emergence of resistance. Similar experiments with HIF-2a inhibitors revealed the acquisition of a HIF-2a mutation, which was subsequently identified in participants of a phase I clinical trial that developed resistance. However, as in humans, resistance to rapamycin in TGs evolved differently. Resistance did not result from the acquisition of an mTOR mutation, even though rapamycin resistance mutations are well established. Furthermore, resistance was transient and was lost with subsequent transplantation or in culture. Unexpectedly, resistance developed despite persistent inhibition of mTORC1 in tumor cells. In contrast, mTORC1 reactivation was observed in non-tumor cells in the TME correlating with the emergence of resistance.

Consistent with this finding, it was further shown herein that the ability of rapamycin to suppress tumor growth was impaired in resistant hosts. As detailed further in the Examples, two traditional approaches to cancer modeling, namely tumor grafting and genetic engineering, were combined to assess the impact of introducing an mTOR resistance mutation into mice. In two of the three TG lines tested, rapamycin failed to inhibit tumor growth, and in the third, tumor growth inhibition by rapamycin was reduced by as much as 5-fold in the resistant host. Collectively, these results show that mTORC1 inhibition in non-tumor cells is essential for the anti-tumor effects of rapalogs.

In the simplest scenario, rapalog-resistant host cells would restore mTORC1 activity in tumor cells. However, this was not the case, and instead it was observed that mTORC1 remained suppressed in tumor cells. These data are most consistent with the host providing a signal that enables tumor cells to grow despite mTORC1 inhibition by rapamycin. This was further analyzed with in vitro experiments, where it was observed that resistant fibroblasts dampened tumor growth inhibition by rapalogs. This was the case only with mTOR-ST fibroblasts, which indicates that the signal provided requires mTORC1 activity. It is noted that in other systems the tumor microenvironment may enable activation of mTORC1 in the tumor cell despite treatment with the mTORC1 inhibitor.

These findings support a model in which mTORC1 inhibition in non-tumor cells is essential for the anti-tumor activity of rapalogs in RCC models and contribute to acquired resistance. These findings explain why mutations in mTOR are not frequently observed in RCC following the acquisition of resistance. Targeting the TME represents a novel therapeutic strategy to enhance the activity of mTORC1 inhibitors in ccRCC and perhaps more broadly in other tumor types.

### III. Methods

Building upon these discoveries, the instant disclosure provides various methods of identifying improved mTORC1 inhibitors, identifying subjects for treatment with mTORC1 inhibitors, and treating subjects with mTORC1 inhibitors.

In one aspect, the present disclosure provides a method of identifying an mTORC1 inhibitor for disease treatment that includes: a) contacting a cell derived from a tumor microenvironment with the mTORC1 inhibitor and b) detecting mTORC1 inhibition in the cell, thereby identifying the mTORC1 inhibitor for disease treatment. In another aspect, the present disclosure provides a method of identifying an mTORC1 inhibitor for disease treatment that includes a) contacting a bystander cell derived from a subject with the mTORC1 inhibitor and b) detecting mTORC1 inhibition in the cell, thereby identifying the mTORC1 inhibitor for disease treatment.

In one embodiment, the detecting is in vitro. In one embodiment, the detecting includes measuring a level of phosphorylation of the mTORC1, S6 kinase, ribosomal S6, or 4EBP, or a combination thereof. In another embodiment, the detecting includes measuring a level of phosphorylated S6 kinase. In an additional embodiment, the detecting includes measuring a decrease in ATP hydrolysis by the mTORC1 isolated from the cell. In a further embodiment, the detecting includes measuring a level of binding of the mTORC1 inhibitor to the mTORC1 isolated from the cell or FKBP12 isolated from the cell.

In one embodiment, the disease is an autoimmune disease, a cancer, a cardiovascular disease, diabetes, an endocrine disorder, an inflammatory disease, kidney disease, a metabolic disorder, muscular atrophy, a neurodegenerative disease, or transplant rejection (e.g., graft-versus-host disease).

In one embodiment, the disease is an autoimmune disease, a cardiovascular disease, diabetes, an endocrine disorder, an inflammatory disease, kidney disease, a metabolic disorder, muscular atrophy, a neurodegenerative disease, or transplant rejection (e.g., graft-versus-host disease). In these aspects, the cell may be a bystander cell, which is defined herein as a cell that is located in the microenvironment of a diseased cell and/or interacts with a diseased cell in the subject, but that does not exhibit the hallmarks of the disease itself.

In one embodiment, the disease is cancer. In another embodiment, the cancer is renal cancer. In an additional embodiment, the renal cancer is clear-cell renal cell carcinoma. In some embodiments, the renal cancer is non-clear-cell renal cell carcinoma (nccRCC). In such aspects, the bystander cell may be a non-cancerous cell derived from a tumor microenvironment. In a further embodiment, the tumor microenvironment cell is an immune cell, stromal cell, or fibroblast. In a particular embodiments, the tumor microenvironment cell is a fibroblast.

In one embodiment, the method further includes detecting mTORC1 inhibition in a cancer cell. In a particular embodiment, the cancer cell is derived from the tumor microenvironment.

Suitable mTORC1 inhibitors are provided herein below. In some aspects, the mTORC1 inhibitor is rapamycin, MTI-31, dactolisib, deforolimus, ridaforolimus, temsirolimus, everolimus, zotarolimus, RAD001, CCI-779, KU-0063794, WYE-354, GSK1059615, AZD8055, nitazoxanide, trokinib, palomid 529, PP121, OSI-027, NU7441, omipalisib, WYE-125132, WYE-687, WAY-600, sapanisertib, torin 1, torin 2, zotarolimus, onatasertib, CZ415, PZR620, or an analogue thereof.

In a further embodiment, the mTORC1 inhibitor is rapamycin or a rapamycin analogue. In a particular embodiment, the rapamycin analogue is deforolimus, ridaforolimus, temsirolimus, everolimus, zotarolimus, RAD001, or CCI-779.

In a further embodiment, the method further includes determining that mTORC1 is inhibited in the cell for at least about 3 days, at least about 5 days, at least about 8 days, at least about 12 days, at least about 15 days, at least about 20 days, at least about 25 days, at least about 30 days, at least about 50 days, at least about 80 days, at least about 120, at least about 150 days, at least about 200 days, at least about 250 days, at least about 300 days, or at least about 500 days. In additional embodiments, the mTORC1 inhibitor is contacted to the cell between about 4 times per day and once every 15 days, between about 4 times per day and once per day, between about once every 1 to 5 days, between about once every 2 to 10 days, between about once every 3 to 15 days, or between about once every 5 to 15 days.

Additional aspects of the present disclosure leverage the discovery that mTORC1 inhibitor resistance in non-cancerous cells can diminish the efficacy of anticancer mTORC1 inhibitor treatment to provide methods for selecting subjects.

Therefore, in certain aspects, the present disclosure provides a method of selecting a subject for treatment of a disease with an mTORC1 inhibitor that includes contacting the mTORC1 inhibitor to a cell derived from a tumor microenvironment of the subject or mTORC1 isolated from the cell; detecting inhibition of mTORC1 in the cell or the mTORC1 isolated from the cell; and selecting the subject for treatment of the disease with the mTORC1 inhibitor. Alternatively, the method may include contacting the mTORC1 inhibitor to a cell derived from a tumor microenvironment of the subject or mTORC1 isolated from the cell; detecting a lack of inhibition of mTORC1 in the cell or the mTORC1 isolated from the cell; and selecting the subject for treatment of the disease with a different mTORC1 inhibitor.

In certain aspects, the present disclosure provides a method of selecting a subject for treatment of a disease with an mTORC1 inhibitor that includes contacting the mTORC1 inhibitor to a cell isolated from the subject or mTORC1 isolated from the cell; detecting inhibition of mTORC1 in the cell or the mTORC1 isolated from the cell; and selecting the subject for treatment of the disease with the mTORC1 inhibitor. In these aspects, the cell may be a bystander cell, which is defined herein as a cell that interacts with a diseased cell in the subject, but that does not exhibit the hallmarks of the disease itself. In some aspects, the cell is an immune cell, a stromal cell, or a fibroblast.

In certain aspects, the present disclosure provides a method of selecting a subject for treatment of a cancer with an mTORC1 inhibitor that includes contacting the mTORC1 inhibitor to a cell derived from a tumor microenvironment of the subject or mTORC1 isolated from the cell; detecting inhibition of mTORC1 in the cell or the mTORC1 isolated from the cell; and selecting the subject for treatment of the disease with the mTORC1 inhibitor. Alternatively, the method may include contacting the mTORC1 inhibitor to a cell derived from a tumor microenvironment of the subject or mTORC1 isolated from the cell; detecting a lack of inhibition of mTORC1 in the cell or the mTORC1 isolated from the cell; and selecting the subject for treatment of the disease with a different mTORC1 inhibitor. In various aspects, the disease may be cancer (e.g., a renal cell carcinoma such as clear cell renal cell carcinoma (cc-RCC) or non-clear cell renal cell carcinoma (ncc-RCC)).

In some aspects, the present disclosure provides a method of selecting a subject for treatment of a cancer with an mTORC1 inhibitor that includes a) i) contacting the mTORC1 inhibitor to a cell derived from a tumor microenvironment of the subject or mTORC1 isolated from the cell and detecting inhibition of mTORC1 in the cell or the mTORC1 isolated from the cell; and/or ii) analyzing a cell derived from a tumor microenvironment from the subject for an mTORC1 inhibitor resistance marker; and b) identifying the subject for treatment with the mTORC1 inhibitor if mTORC1 inhibition is detected and/or if the mTORC1 inhibitor resistance marker is not detected.

Also provided are methods of treating a subject with an mTORC1 inhibitor, the methods comprising administering the mTORC1 inhibitor to the subject, wherein the subject has been identified for treatment with the mTORC1 inhibitor according to any method provided herein.

Also provided are methods of treating a cancer in a subject in need thereof with an mTORC1 inhibitor, the method comprising: a) contacting the mTORC1 inhibitor to a cell derived from a tumor microenvironment of the subject or mTORC1 isolated from the cell and detecting inhibition of mTORC1 in the cell or the mTORC1 isolated from the cell; and/or analyzing a cell derived from a tumor microenvironment from the subject for an mTORC1 inhibitor resistance marker; and b) administering the mTORC1 inhibitor to the subject if mTORC1 inhibition is detected and/or if the mTORC1 inhibitor resistance marker is not detected.

In some embodiments, the methods disclosed herein include contacting the mTORC1 inhibitor to a cell and detecting the inhibition of the mTORC1 in the cell, for example by detecting diminished mTORC1 signaling in the cell or a lysate derived from the cell. In other embodiments, the method includes contacting the mTORC1 inhibitor to the mTORC1 isolated from a cell and detecting the inhibition of the mTORC1 isolated from the cell, for example by measuring a rate of ATP hydrolysis or substrate phosphorylation by mTORC1. In some aspects, therefore the methods can comprise 1) measuring a decrease in ATP hydrolysis by the mTORC1 isolated from the cell; and/or 2) measuring a level of binding of the mTORC1 inhibitor to the mTORC1 isolated from the cell or FKBP12 isolated from the cell; and/or 3) precipitating the mTORC1 isolated from the cell and measuring activity of the mTORC1 in the presence of a protein substrate and ATP.

As used herein, the term “mTORC1 inhibitor resistance” encompasses diminished mTORC1 inhibitor activity (e.g., rapamycin or rapalog FRB or FKBP12 binding affinity), mTORC1 constitutive activity, or modulated expression of proteins involved in mTORC1 signaling. As used herein, the term “mTORC1 inhibitor resistance marker” encompasses mTORC1 constitutive activity, or modulated expression of proteins involved in mTORC1 signaling, or any combination of p-S6K, p-S6, p-4EBP1, and p-ULK1.

In any of the aspects described herein, detection of mTORC1 inhibition and/or detection of the mTORC1 inhibitor resistance marker can be performed in vivo or in vitro. In some embodiments, detecting mTORC1 inhibition includes measuring activity of mTORC1. For example, the detecting can include isolating mTORC1 from a tumor microenvironment cell, contacting the mTORC1 with a substrate or plurality of substrates, and measuring ATP hydrolysis in the presence and optionally in the absence of the mTORC1 inhibitor. Numerous methods for measuring ATP hydrolysis are known in the art, and include colorimetric detection with an ATPase assay, fluorescence detection with a magnesium green assay, and electrochemical detection of ATP. The detecting can also include optionally collecting or isolating mTORC1 from the cell, contacting the mTORC1 with a substrate or plurality of mTORC1 substrates, and measuring phosphorylation of one or more substrates as outlined further herein. Non-limiting examples of methods for measuring phosphorylation include immunofluorescent (or immunohistochemical) detection (e.g., with a fluorescent antibody specific for a specific phosphorylated protein), enzyme linked immunosorbent assay (ELISA), Western blotting, competitive binding assay, radioimmunoassay, analysis with a reverse phosphorylation protein array (RPPA), and mass spectrometric detection (e.g., with a molecular dissociation assay). Additionally, phosphorylation measurements may be performed with a commercially available kit, such as an AlphaLISA SUREFIRE ULTRA phospho-mTOR (Ser2448) Assay Kit. mTOR (e.g., mTORC1) can be selectively precipitated (e.g., immunohistochemically) or collected from cell lysate. Alternatively, mTORC1 inhibition can be measured within a live cell. In particular embodiments, detecting mTORC1 inhibition includes measuring a decrease in ATP hydrolysis by the mTORC1 isolated from the cell.

Alternatively, or in addition thereto, detecting mTORC1 inhibition can include detecting a level of binding between an mTOR inhibitor and mTORC1, between the mTOR inhibitor and an mTOR substrate protein (e.g., FK506-binding protein 12 (FKBP12) or Eukaryotic translation initiation factor 4E-binding protein 1 (4EBP)), or a combination thereof. Non-limiting examples of inhibitor binding assays amenable to the present disclosure include competitive inhibition assays, fluorescent inhibitor assays, and mTOR substrate phosphorylation assays as described herein. In a particular embodiment, the detecting includes measuring a level of binding of the mTOR inhibitor to the mTOR or FKBP12 isolated from the cell.

In some embodiments, the detecting includes measuring a level of phosphorylation of the mTORC1 or a downstream signaling protein. For example, the detecting can include measuring a level of phosphorylation of the mTORC1 or a level of phosphorylation of S6 kinase, ribosomal S6, or 4EBP, or a combination thereof. In certain embodiments, the detecting includes measuring S6 kinase (S6K)/ribosomal S6 effector pathway activity. In particular embodiments, the detecting includes measuring a level of phosphorylated S6 kinase.

As further detailed herein, identifying a lack of mTORC1 inhibitor resistance in a tumor microenvironment cell in a subject can thus designate that subject as a likely responder to mTORC1 inhibitor therapy.

Therefore, in another aspect, the present disclosure provides a method of selecting a subject for treatment of a disease with an mTORC1 inhibitor including: analyzing a cell from a tumor microenvironment of the subject for a mTORC1 inhibitor resistance marker; and selecting the subject for treatment of the disease with the mTORC1 inhibitor. In another aspect, the present disclosure provides a method of selecting a subject for treatment of a cancer with an mTORC1 inhibitor including: analyzing a cell from a tumor microenvironment of the subject for a mTORC1 inhibitor resistance marker; and selecting the subject for treatment of the disease with the mTORC1 inhibitor. The method can include determining the absence of the mTORC1 inhibitor resistance marker in the subject, for example by genetically profiling the subject for one or more genetic markers associated with mTOR inhibitor resistance. In some embodiments, the mTORC1 inhibitor resistance marker is p-S6K, p-S6, p-4EBP1, p-ULK1, or a combination thereof. In some embodiments, the mTORC1 inhibitor resistance marker comprises mTORC1 constitutive activity, or modulated expression of proteins involved in mTORC1 signaling. In some aspects, the methods provided herein can include genetically profiling the subject for one or more genetic markers associated with mTOR inhibitor resistance (e.g., a genetic mutation in a gene encoding for mTOR or any other protein in mTORC1). In some embodiments, determining the absence of a resistance marker for the mTORC1 inhibitor (e.g., the mTORC1 inhibitor) includes DNA or mRNA profiling.

In another aspect, the present disclosure provides a method of selecting a subject for treatment of a disease with an mTORC1 inhibitor, the method including: analyzing a cell from a tumor microenvironment of the subject for a mTORC1 inhibitor resistance marker; and selecting the subject for treatment of the disease with the mTORC1 inhibitor if the mTORC1 inhibitor resistance marker is not detected. In another aspect, the present disclosure provides a method of selecting a subject for treatment of a cancer with an mTORC1 inhibitor, the method including: analyzing a cell from a tumor microenvironment of the subject for a mTORC1 inhibitor resistance marker; and selecting the subject for treatment of the disease with the mTORC1 inhibitor if the mTORC1 inhibitor resistance marker is not detected. In some embodiments, the mTORC1 inhibitor resistance marker is p-S6K, p-S6, p-4EBP1, p-ULK1, or a combination thereof. In some embodiments, the mTORC1 inhibitor resistance marker comprises mTORC1 constitutive activity, or modulated expression of proteins involved in mTORC1 signaling. In some aspects, the methods of analyzing the cell for an mTORC1 inhibitor resistance marker comprises profiling the subject for a genetic mutation. In various aspects, the genetic mutation is associated with increased or constitutive mTORC1 activity. In some aspects, the methods provided herein can include genetically profiling the subject for one or more genetic markers associated with mTOR inhibitor resistance (e.g., a genetic mutation in a gene encoding for mTOR or any other protein in mTORC1, especially a genetic mutation associated with increased or constitutive mTORC1 activity). In some embodiments, determining the absence of a resistance marker for the mTORC1 inhibitor (e.g., the mTORC1 inhibitor) includes DNA or mRNA profiling.

In another aspect, the present disclosure provides a method of treating a subject for a cancer with an mTORC1 inhibitor, the method including: analyzing a cell from a tumor microenvironment of the subject for a mTORC1 inhibitor resistance marker; and selecting the subject for treatment of the disease with the mTORC1 inhibitor if the mTORC1 inhibitor resistance marker is not detected. In another aspect, the present disclosure provides a method of treating a subject for a cancer with an mTORC1 inhibitor, the method including: analyzing a cell from a tumor microenvironment of the subject for a mTORC1 inhibitor resistance marker; and selecting the subject for treatment of the disease with the mTORC1 inhibitor if the mTORC1 inhibitor resistance marker is not detected. In some embodiments, the mTORC1 inhibitor resistance marker is p-S6K, p-S6, p-4EBP1, p-ULK1, or a combination thereof. In some embodiments, the mTORC1 inhibitor resistance marker comprises mTORC1 constitutive activity, or modulated expression of proteins involved in mTORC1 signaling. In some aspects, the methods of analyzing the cell for an mTORC1 inhibitor resistance marker comprises profiling the subject for a genetic mutation. In various aspects, the genetic mutation is associated with increased or constitutive mTORC1 activity. In some aspects, the methods provided herein can include genetically profiling the subject for one or more genetic markers associated with mTOR inhibitor resistance (e.g., a genetic mutation in a gene encoding for mTOR or any other protein in mTORC1, especially a genetic mutation associated with increased or constitutive mTORC1 activity). In some embodiments, determining the absence of a resistance marker for the mTORC1 inhibitor (e.g., the mTORC1 inhibitor) includes DNA or mRNA profiling.

In some embodiments, any of the methods of selecting the subject may further include: d) repeating the detecting inhibition of mTORC1 in the cell or the mTORC1 isolated from the cell subsequently to the selecting the subject. In some embodiments, d) is performed about 3 days to 600 days, about 3 days to 15 days, about 10 days to 50 days, about 20 days to 100 days, about 40 days to 200 days, about 60 days to 300 days, or about 120 days to 600 days after c). In some embodiments, the method further includes continuing treatment with the mTORC1 inhibitor. In other embodiments, upon detecting a lack of inhibition of the mTORC1 in the cell or the mTORC1 isolated from the cell subsequently to the selecting subject, the method includes ceasing treatment with the mTORC1 inhibitor.

In some embodiments, any of the methods of treating the subject may further include: d) repeating detecting inhibition of mTORC1 in the cell or the mTORC1 isolated from the cell and/or repeating analyzing a cell derived from a tumor microenvironment from the subject for an absence of the mTORC1 inhibitor resistance marker and e) continuing treatment with the mTORC1 inhibitor if mTORC1 inhibition is detected and/or the mTORC1 inhibitor resistance marker is absent. In some embodiments, d) is performed about 3 days to 600 days, about 3 days to 15 days, about 10 days to 50 days, about 20 days to 100 days, about 40 days to 200 days, about 60 days to 300 days, or about 120 days to 600 days after c).

While many cancers do not exhibit mTOR mutations, mTOR activity is commonly upregulated in human cancers, and is therefore an important target for cancer treatment. mTOR inhibition can reverse the effects of increased mTOR activity, and, in certain cancers, can arrest cell growth and proliferation. Accordingly, in a particular aspect, the present disclosure provides a method of selecting a subject for treatment of a cancer with an mTORC1 inhibitor that includes contacting the mTORC1 inhibitor to mTORC1 from a non-cancerous cell or mTORC1 isolated from a non-cancerous cell from the subject; detecting inhibition of mTORC1 in the non-cancerous cell or the mTORC1 isolated from the cell; and selecting the subject for treatment of the cancer with the mTORC1 inhibitor. In view of this, also provided is a method of treating a cancer in a subject with an mTORC1 inhibitor, the method comprising contacting the mTORC1 inhibitor to mTORC1 from a non-cancerous cell or mTORC1 isolated from a non-cancerous cell from the subject; detecting inhibition of mTORC1 in the non-cancerous cell or the mTORC1 isolated from the cell; and administering the mTORC1 inhibitor to the subject.

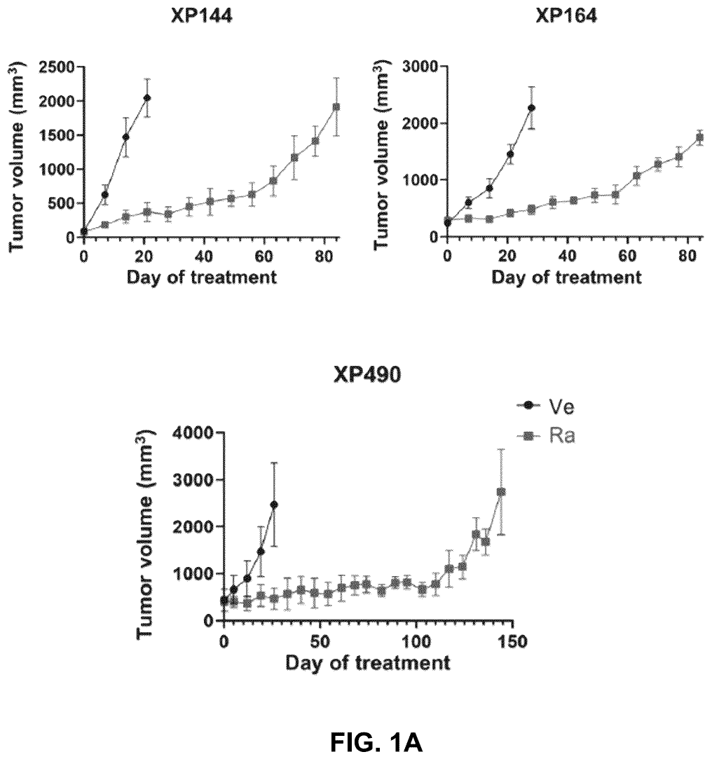

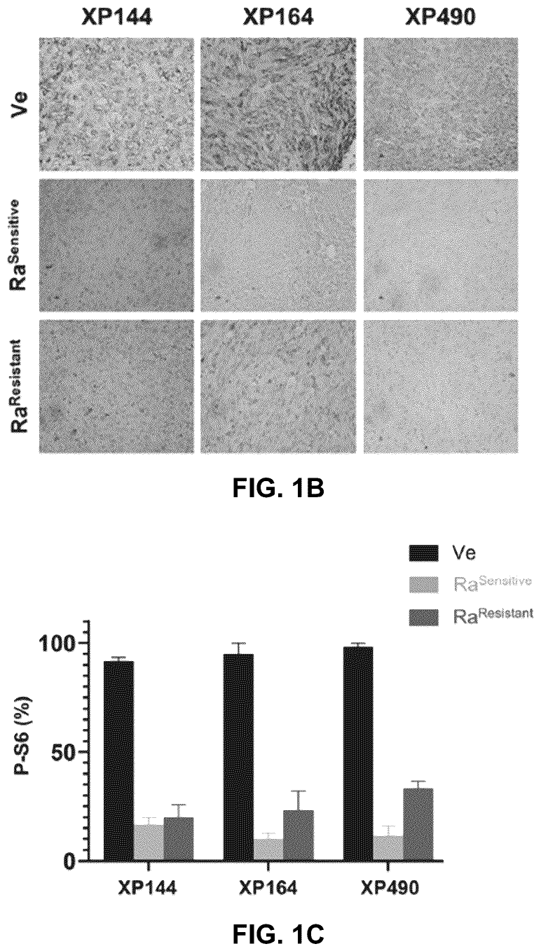

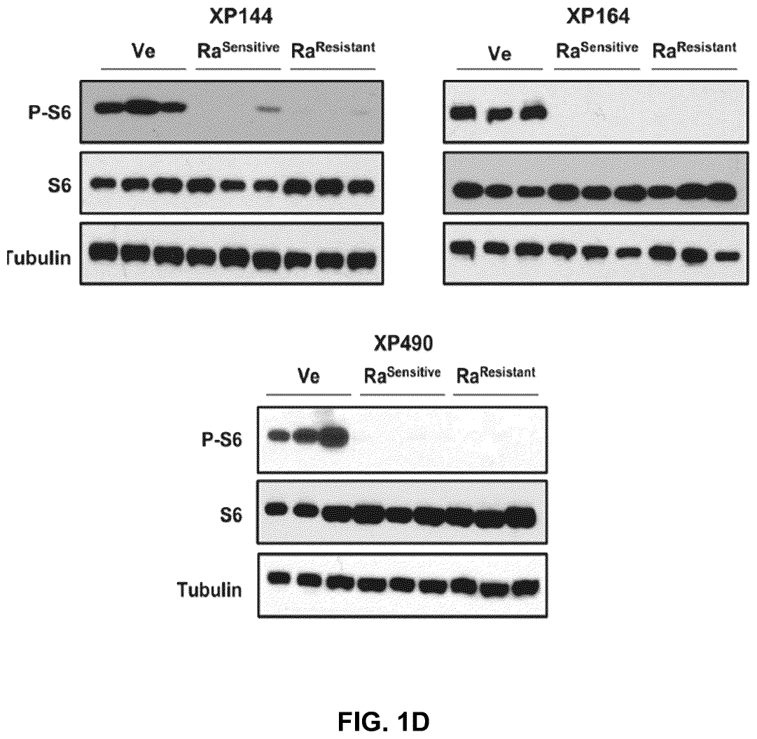

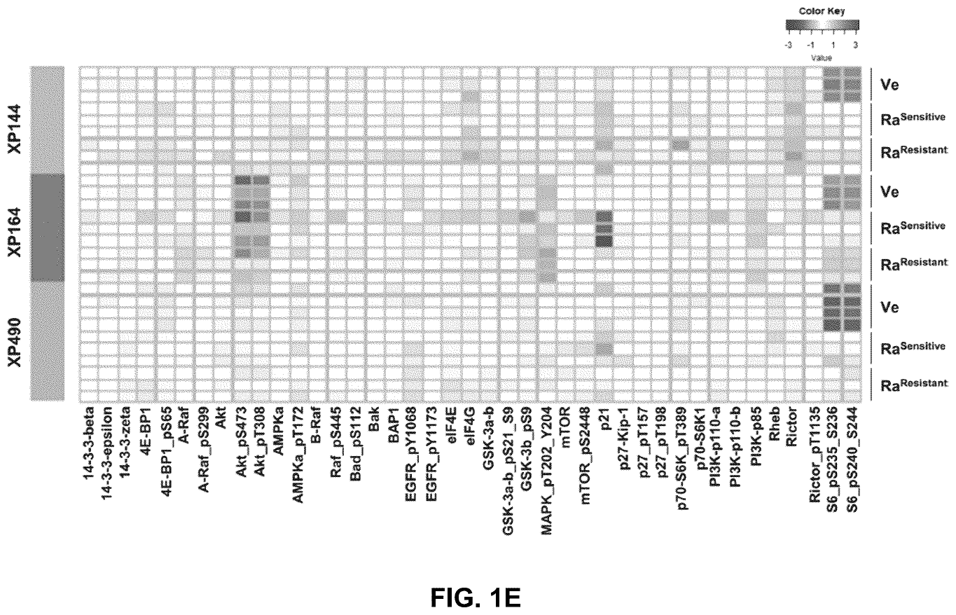

In some embodiments, the disease is cancer. In particular embodiments, the disease is renal cancer. In some embodiments, the cancer is renal cell carcinoma (RCC), which is a particular focus and exemplary embodiment of the present disclosure. RCC is among the most prevalent forms of cancer, and is the most common form of kidney cancer in adults. RCC often coincides with activated mTOR signaling, suggesting a possible role of mTOR signaling in RCC development and rendering RCC treatment challenging with conventional chemotherapeutics. As demonstrated herein (for example in FIG. 1A), mTOR (e.g., mTORC1) inhibition can greatly reduce RCC tumor growth.

In specific embodiments, the cancer is clear-cell RCC (ccRCC). ccRCC accounts for about three-fourths of all RCC cases and is a particularly aggressive form of cancer that is associated with low survival rates. While mTOR inhibition is important for ccRCC treatment, acquired mTOR inhibitor resistance can render mTOR inhibitor treatment of ccRCC challenging and ineffective in many subjects.

In some embodiments, the renal cancer is non-clear-cell renal cell carcinoma (nccRCC).

In some embodiments, the cell (e.g., the non-cancerous cell or cancer cell analyzed for mTORC1 inhibition) is derived from a tumor microenvironment. Cancer cells participate in extensive bidirectional signaling with surrounding non-cancerous cells. This signaling can affect their growth, development, and ability to escape immune detection. It was nonetheless surprisingly demonstrated herein that tumor resistance to rapamycin can be associated with persistent mTORC1 activation in tumor microenvironment cells such as myofibroblasts (e.g., as detailed in Example 8), suggesting that mTORC1 inhibition in cancer cells and tumor microenvironment cells is required for anticancer activity. In some embodiments, the tumor microenvironment cell is an immune cell, a stromal cell, or a fibroblast. In some embodiments, the tumor microenvironment cell is a fibroblast or a myofibroblast.

In certain aspects, the present disclosure provides a method of selecting a subject for treatment of a disease with an mTORC1 inhibitor that includes contacting the mTORC1 inhibitor to a cell isolated from the subject or mTORC1 isolated from the cell; detecting inhibition of mTORC1 in the cell or the mTORC1 isolated from the cell; and selecting the subject for treatment of the disease with the mTORC1 inhibitor. In some aspects, the cell is an immune cell, a stromal cell, or a fibroblast. In some aspects, the cell is a bystander cell, which is defined herein as a cell that interacts with a diseased cell in the subject, but that does not exhibit the hallmarks of the disease itself.

Also provided are methods of treating a disease with an mTORC1 inhibitor that includes contacting the mTORC1 inhibitor to a cell isolated from the subject or mTORC1 isolated from the cell; detecting inhibition of mTORC1 in the cell or the mTORC1 isolated from the cell; and treating the subject with the mTORC1 inhibitor. In some aspects, the cell is an immune cell, a stromal cell, or a fibroblast. In some aspects, the cell is a bystander cell, which is defined herein as a cell that interacts with a diseased cell in the subject, but that does not exhibit the hallmarks of the disease itself.

In any of the embodiments herein, the disease is an autoimmune disease, a cancer, a cardiovascular disease, diabetes, an endocrine disorder, an inflammatory disease, kidney disease, a metabolic disorder, muscular atrophy, a neurodegenerative disease, or transplant rejection (e.g., graft-versus-host disease). In certain embodiments, the disease is a cancer (e.g., a renal cell cancer such as clear-cell renal cell carcinoma (ccRCC) or non-clear-cell renal cell carcinoma (nccRCC)).

In a further aspect, the present disclosure provides a method of selecting a treatment method for treating a subject with a disease in need thereof that includes: contacting the mTORC1 inhibitor to a cell derived from a tumor microenvironment of the subject or mTORC1 isolated from the cell; detecting activity of mTORC1 in the cell or the mTORC1 isolated from the cell; and administering the mTORC1 inhibitor to the subject if the activity of the mTORC1 in the cell or the mTORC1 isolated from the cell is inhibited. In certain embodiments, the disease is a cancer (e.g., a renal cell cancer such as clear-cell renal cell carcinoma (ccRCC) or non-clear-cell renal cell carcinoma (nccRCC)).

In measuring mTORC1 activity, the method can include determining that the mTORC1 in the cell from the subject is inhibited by the mTORC1 inhibitor, thereby indicating that the subject is a likely responder to mTORC1 inhibitor therapy. In some embodiments, the method includes determining whether to continue an mTORC1 inhibitor therapy presently being administered to the subject.

As it was determined herein that bystander cells can confer resistance to mTOR inhibitors, and furthermore that bystander cell mTORC1 inhibitor resistance can be temporary, in another aspect, the present disclosure provides a method of treating a subject with a disease (e.g., a disease treatable with an mTORC1 inhibitor) that includes: administering an mTORC1 inhibitor to the subject for a first time period; ceasing administration of the mTORC1 inhibitor for a second time period following the first time period; and resuming administration of the mTORC1 inhibitor following the second time period for a third time period.

In some embodiments, the first time period is about 1 to 5 weeks, about 1 to 10 weeks, about 2 to 10 weeks, about 2 to 15 weeks, about 2 to 20 weeks, about 5 to 20 weeks, about 5 to 30 weeks, about 10 to 30 weeks, or about 20 to 40 weeks. In some embodiments, the second time period is about 2 to 60 days, about 2 to 5 days, about 2 to 10 days, about 2 to 20 days, about 5 to 20 days, about 5 to 40 days, about 10 to 40 days, about 10 to 60 days, about 20 to 60 days, about 30 to 60 days, or about 40 to 60 days. In some embodiments, the third time period is about 1 to 5 weeks, about 1 to 10 weeks, about 2 to 10 weeks, about 2 to 15 weeks, about 2 to 20 weeks, about 5 to 20 weeks, about 5 to 30 weeks, about 10 to 30 weeks, or about 20 to 40 weeks.

Also provided are methods of treating a subject with a cancer, the method comprising a) administering an mTORC1 inhibitor to the subject for a first time period; b) ceasing administration of the mTORC1 inhibitor for a second time period following the first time period if the disease has been determined to be resistant to the mTORC1 inhibitor; and c) resuming administration of the mTORC1 inhibitor following the second time period for a third time period. In these aspects, the cancer may have been determined to be resistant to the mTORC1 inhibitor when a cell derived from the tumor microenvironment from the subject is resistant to mTORC1 inhibition and/or has an mTORC1 inhibitor resistance marker. Accordingly, in some aspects, the method further comprises measuring the resistance to the mTORC1 inhibitor and/or detecting an mTORC1 inhibitor resistance marker in a cell derived from the tumor microenvironment from the subject about every 1, 2, 3, 4, 5, 6, 7, 8, 9, 10, 12, 15, 20, 25, 30, 40, 50, or 60 days. In these or related aspects, the second time period may be about 2 to 60 days, about 2 to 5 days, about 2 to 10 days, about 2 to 20 days, about 5 to 20 days, about 5 to 40 days, about 10 to 40 days, about 10 to 60 days, about 20 to 60 days, about 30 to 60 days, or about 40 to 60 days.

In a further aspect, the present disclosure provides a method of selecting a treatment method for treating a subject with a disease in need thereof that includes: contacting the mTORC1 inhibitor to a cell derived from a tumor microenvironment of the subject or mTORC1 isolated from the cell; detecting activity of mTORC1 in the cell or the mTORC1 isolated from the cell; and administering the mTORC1 inhibitor to the subject if the activity of the mTORC1 in the cell or the mTORC1 isolated from the cell is inhibited. In a further aspect, the present disclosure provides a method of treating a subject with a disease that includes: a) analyzing a cell derived from a tumor microenvironment from the subject for a mTORC1 inhibitor resistance marker; and b) administering to the subject an mTORC1 inhibitor.

The mTORC1 inhibitor resistance marker can be measured in tumor microenvironment cells from the subject about every 1, 2, 3, 4, 5, 6, 7, 8, 9, 10, 12, 15, 20, 25, 30, 40, 50, or 60 days, and treatment with the mTOR inhibitor (e.g., the mTORC1 inhibitor) can continue until mTORC1 inhibitor resistance is detected in a tumor microenvironment cell. In some embodiments, the subject is selected for the mTORC1 inhibitor therapy with a method disclosed herein.

In further aspects, a method is also provided of determining the role of the tumor microenvironment in response to a therapy, the method comprising i) transplanting a first tumor or tumor cell into a first immunocompromised mammal comprising a mutation and a second immunocompromised mammal that does not comprise the mutation; ii) administering the therapy to the first and second immunocompromised mammals; and iii) comparing a response of the tumor or tumor cell in the first and second immunocompromised mammals; thereby determining the role of the tumor microenvironment in response to the therapy.

In various aspects, the first and second immunocompromised mammals are mice. In some aspects, the first and second immunocompromised mammals are NOD or SCID mice.

In various aspects, the mutation is an mTOR mutation. In some aspects, the mutation is an EGFR (epidermal growth factor receptor) mutation. In some aspects, the mutation is an Abl kinase mutation. In various aspects, the mutation is a Ret mutation (i.e., a RET proto-oncogene mutation).

In various aspects, the therapy comprises an mTOR inhibitor (e.g., an mTORC1 inhibitor). In various aspects, the therapy comprises an EGFR inhibitor. In various aspects, the therapy comprises an Abl kinase inhibitor. In various aspects, the therapy comprises a Ret inhibitor.

In some aspects, the mutation is an mTOR mutation and the therapy comprises an mTOR inhibitor (e.g., an mTORC1 inhibitor). In some aspects, the mutation is an EGFR (epidermal growth factor receptor) mutation, and the therapy comprises an EGFR inhibitor. In some aspects, the mutation is an Abl kinase mutation, and the therapy comprises an Abl kinase inhibitor. In some aspects, the mutation is a Ret mutation (i.e., a RET proto-oncogene mutation) and the therapy comprises a Ret inhibitor.

Suitable mTORC1 inhibitors are provided herein below. For example, the mTORC1 inhibitor can comprise rapamycin, MTI-31, dactolisib, deforolimus, ridaforolimus, temsirolimus, everolimus, zotarolimus, RAD001, CCI-779, KU-0063794, WYE-354, GSK1059615, AZD8055, nitazoxanide, trokinib, palomid 529, PP121, OSI-027, NU7441, omipalisib, WYE-125132, WYE-687, WAY-600, sapanisertib, torin 1, torin 2, zotarolimus, onatasertib, CZ415, PZR620, or an analogue thereof. In some embodiments, the mTORC1 inhibitor is rapamycin or an analogue thereof. In some embodiments, the mTORC1 inhibitor is temsirolimus or everolimus.

Suitable EGFR inhibitors are known in the art and may include, but are not limited to erlotinib, osimertinib, neratinib, cetuximab, gefitinib, necitumumab, vandetanib, mobocertinib, lapatinib, dacomitinib, panitumumab, and lazertinibib. Accordingly, the EGFR inhibitors may comprise erlotinib, osimertinib, neratinib, cetuximab, gefitinib, necitumumab, vandetanib, mobocertinib, lapatinib, dacomitinib, panitumumab, lazertinibib, or an analogue thereof.

Suitable Abl kinase inhibitors are known in the art and may include but are not limited to: imatinib, dasatinib, nilotinib, bosutinib, panatinib, and asciminib. Accordingly, the Abl kinase inhibitor may comprise imatinib, dasatinib, nilotinib, bosutinib, panatinib, asciminib, or an analogue thereof.

Suitable Ret inhibitors are known in the art and may include but are not limited to: pralsetinib and selpercatinib. Accordingly, the Ret inhibitor may comprise pralsetinib, selpercatinib, or an analogue thereof.

Any other or future mTORC1, Abl kinase, EGFR, and Ret inhibitors are also contemplated for use in the present disclosure.

Numerous mTORC1 inhibitors are suitable for use in the present disclosure. In some embodiments, the mTORC1 inhibitor selected from rapamycin, MTI-31, dactolisib, deforolimus, ridaforolimus, temsirolimus, everolimus, zotarolimus, RAD001, CCI-779, KU-0063794, WYE-354, GSK1059615, AZD8055, nitazoxanide, trokinib, palomid 529, PP121, OSI-027, NU7441, omipalisib, WYE-125132, WYE-687, WAY-600, sapanisertib, torin 1, torin 2, zotarolimus, onatasertib, CZ415, PZR620, or an analogue thereof.

In some embodiments, the mTORC1 inhibitor is rapamycin or a rapamycin analogue (hereinafter referred to interchangeably with the term “rapalogs”). Rapamycin and rapalogs inhibit mTORC1 by antagonizing the FK506 Rapamycin Binding (FRB) domain of mTORC1. Rapalogs include, deforolimus, ridaforolimus, temsirolimus, everolimus, zotarolimus, RAD001, and CCI-779. In some embodiments, the mTORC1 inhibitor (e.g., rapamycin or a rapalog) binds to the FK506 Rapamycin Binding (FRB) domain of mTOR kinase. In some embodiments, the mTORC1 inhibitor is a biomolecule or compound that inhibits the activity of FKBP12 or FKBP51. In some embodiments, the mTORC1 inhibitor is temsirolimus or everolimus. In some embodiments, the mTORC1 inhibitor is rapamycin, deforolimus, ridaforolimus, temsirolimus, everolimus, zotarolimus, RAD001, CCI-779 or an analogue thereof

In some embodiments, the mTORC1 inhibitor is administered in combination with an additional therapeutic agent (e.g., an anti-cancer therapy or agent). Suitable anti-cancer agents or therapies include, but are not limited to, immunostimulators, immunotherapies, oncolytic viruses, polysaccharides, neoantigens, chemotherapies, targeted therapies, hormone therapies, radiotherapy, or surgery and are described further below.

### Immunostimulators

The anti-cancer therapy or agent may be an immunostimulator. The term “immunostimulator” as used herein refers to a compound that can stimulate an immune response in a subject, and may include an adjuvant. In some embodiments, an immunostimulator is an agent that does not constitute a specific antigen, but can boost the strength and longevity of an immune response to an antigen. Such immunostimulators may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherichia coli, Salmonella minnesota, Salmonella typhimurium, or Shigella flexneri or specifically with MPL (AS04), MPL A of above-mentioned bacteria separately, saponins, such as QS-21, Quil-A, ISCOMs, ISCOMATRIX, emulsions such as MF59, Montanide, ISA 51 and ISA 720, AS02 (QS21+squalene+MPL.), liposomes and liposomal formulations such as ASO1, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments.

The additional anti-cancer therapy may comprise an agonist for pattern recognition receptors (PRR), including, but not limited to Toll-Like Receptors (TLRs), specifically TLRs 2, 3, 4, 5, 7, 8, 9 and/or combinations thereof. The additional anti-cancer therapy may comprise agonists for Toll-Like Receptors 3, agonists for Toll-Like Receptors 7 and 8, or agonists for Toll-Like Receptor 9; preferably the recited immunostimulators comprise imidazoquinolines; such as R848; adenine derivatives, such as those disclosed in U.S. Pat. No. 6,329,381, U.S. Published Patent Application 2010/0075995, or WO 2010/018132; immunostimulatory DNA; or immunostimulatory RNA. In some embodiments, the additional anti-cancer therapies also may comprise immunostimulatory RNA molecules, such as but not limited to dsRNA, poly 1:C or poly I:poly C12U (available as Ampligen.RTM., both poly 1:C and poly I:polyC12U being known as TLR3 stimulants), and/or those disclosed in F. Heil et al., “Species-Specific Recognition of Single-Stranded RNA via Toll-like Receptor 7 and 8” Science 303(5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E.

Uhlmann et al., “Modified oligoribonucleotide analogs with enhanced immunostimulatory activity” U.S. Pat. Appl. Publ. US 2006241076; G. Lipford et al., “Immunostimulatory viral RNA oligonucleotides and use for treating cancer and infections” WO 2005097993 A2; G. Lipford et al., “Immunostimulatory G,U-containing oligoribonucleotides, compositions, and screening methods” WO 2003086280 A2. In some embodiments, an additional anti-cancer therapy may be a TLR-4 agonist, such as bacterial lipopolysaccharide (LPS), VSV-G, and/or HMGB-1. In some embodiments, additional therapies may comprise TLR-5 agonists, such as flagellin, or portions or derivatives thereof, including but not limited to those disclosed in U.S. Pat. Nos. 6,130,082, 6,585,980, and 7,192,725.

In some embodiments, additional anti-cancer therapies may be proinflammatory stimuli released from necrotic cells (e.g., urate crystals). In some embodiments, additional anti-cancer therapies may be activated components of the complement cascade (e.g., CD21, CD35, etc.). In some embodiments, additional anti-cancer therapies may be activated components of immune complexes. Additional anti-cancer therapies also include complement receptor agonists, such as a molecule that binds to CD21 or CD35. In some embodiments, the complement receptor agonist induces endogenous complement opsonization of the synthetic nanocarrier. In some embodiments, immunostimulators are cytokines, which are small proteins or biological factors (in the range of 5 kD-20 kD) that are released by cells and have specific effects on cell-cell interaction, communication and behavior of other cells. In some embodiments, the cytokine receptor agonist is a small molecule, antibody, fusion protein, or aptamer.

### Immunotherapies

In some embodiments, the additional anti-cancer therapy comprises a cancer immunotherapy. Cancer immunotherapy (sometimes called immuno-oncology, abbreviated 10) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g., carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies are known in the art, and some are described below.

### Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### Dendritic Cell Therapy

The additional anti-cancer therapy may comprise dendritic cells. Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (i.e. a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

### Car-T Cell Therapy

The additional anti-cancer therapy may comprise a chimeric antigen receptor (CAR). CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

### Cytokine Therapy

The additional anti-cancer therapy may comprise cytokines. Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### Adoptive T-Cell Therapy

The additional anti-cancer therapy may comprise adoptive T cell therapy. Adoptive T cell therapy is a form of passive immunization by the transfusion of T-cells (adoptive cell transfer). They are found in blood and tissue and usually activate when they find foreign pathogens. Specifically, they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### Checkpoint Inhibitors and Combination Treatment

In some embodiments, the additional anti-cancer immunotherapy comprises immune checkpoint inhibitors. Certain embodiments are further described below.

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Additional anti-cancer therapies of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2. In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1.

In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DClg, is a PDL2-Fc fusion soluble receptor described in WO2010/027827 and WO2011/066342. Additional anti-cancer PD-1 inhibitors include MED10680, also known as AMP-514, and REGN2810.

In some embodiments, the immune checkpoint inhibitor is a PDL1 inhibitor such as Durvalumab, also known as MED14736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

Another immune checkpoint that can be targeted in the methods provided herein as an additional anti-cancer therapy is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art.

Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO2001/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### Oncolytic Virus

In some embodiments, the additional anti-cancer therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy.

### Polysaccharides

In some embodiments, the additional anti-cancer therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

### Neoantiqens

In some embodiments, the additional anti-cancer therapy comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

### Chemotherapies

In some embodiments, the additional anti-cancer therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain embodiments. In some embodiments, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### Targeted Therapies

In some embodiments, the additional anti-cancer therapy comprises a targeted therapy. Targeted therapies are drugs or other substances that block the growth and spread of cancer by interfering with specific molecules (“molecular targets”) that are involved in the growth, progression, and/or spread of cancer. Targeted cancer therapies are sometimes called “molecularly targeted drugs,” “molecularly targeted therapies,” “precision medicines,” or similar names. Non-limiting examples of targeted therapies include hormone therapies, signal transduction inhibitors, gene expression modulators, apoptosis inducers, angiogenesis inhibitors, immunotherapies, toxin delivery molecules, and the like. In particular embodiments, the targeted therapy may be a poly ADP ribose polymerase (PARP) inhibitor (e.g., niraparib). PARP (e.g., PARP-1 and/or PARP-2) inhibitors are well known in the art (e.g., Olaparib, ABT-888, BSI-201, BGP-15, INO-1001, PJ34, 3-aminobenzamide, 4-amino-1,8-naphthalimide, 6(5H)-phenanthridinone, benzamide, NU1025).

### Hormone Therapy

In some embodiments, the additional anti-cancer therapy comprises hormone therapy. In some embodiments, one or more anticancer therapies may be hormonal therapy, Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

### Radiotherapy

In some embodiments, the additional anti-cancer therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5,6, 7, 8, 9, 10, 11, 12, 13, 14, 15, 16, 17, 18, 19,20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### Surgery

The additional anti-cancer therapy may comprise surgery. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

The following examples are provided to further illustrate the embodiments of the present disclosure, but are not intended to limit the scope of the disclosure. While they are typical of those that might be used, other procedures, methodologies, or techniques known to those skilled in the art may alternatively be used.

### Examples

### Example 1: Experimental Methods

### Tumorgrafts

Tumor samples were collected from patients under IRB-approved protocol as previously described. 6-to-8-week-old NOD/SCID mice originally obtained from the UT Southwestern Breeding Core were implanted subcutaneously with patient-derived TGs. Calipers were used to measure tumor dimensions. Volume was calculated by multiplying width, length, and height, thereby minimizing the potential bias of disproportionately weighing one particular dimension (at the cost of overestimating tumor volumes). Once tumor volumes reached ˜100-350 mm³, mice were treated with rapamycin or vehicle as a control. Rapamycin (LC Laboratories) was dissolved in ethanol (Pharmco, Greenfield Global) and combined with a solution of 5% polyethylene glycol (Sigma) and 5% Tween 80 (Sigma) in 5% dextrose (Sigma). Rapamycin was delivered intraperitoneally (IP) every 48 hours at a dose of 0.5 mg/kg. Tumors were measured weekly. As indicated, vehicle-treated tumors and rapamycin-sensitive tumors were harvested around 28 days. A subset of mice was continually treated with rapamycin until the tumor volume was comparable to that of the vehicle-treated tumors, and these were considered to be rapamycin-resistant. In accordance with UT Southwestern Institutional Animal Care and Use Committee policies, animals were euthanized once tumor diameters were larger than 2 cm.

For subsequent transplantations, rapamycin-resistant tumors were harvested, minced into pieces (˜4 mm in diameter) and transplanted subcutaneously into additional cohorts of NOD/SCID mice.

CRISPR-Cas9 editing of NOD/SCID mice

Four sgRNAs were designed using the MIT CRISPR design tool: sgRNA1, 5′-acaagcgagatgcctcttcc-3′(SEQ ID NO: 2); sgRNA2, 5′-gaggcatctcgcttgtactt-3′ (SEQ ID NO: 3); sgRNA3, 5′-aggcatctcgcttgtacttt-3′ (SEQ ID NO: 4); and sgRNA4, 5′-ggcatctcgcttgtactttg-3′ (SEQ ID NO: 5). The sgRNAs were first tested in mouse NIH/3T3 cells using the surveyor assay by SURVEYOR Mutation Detection Kit (Integrated DNA Technologies). Using this strategy, we identified sgRNA1 and sgRNA4 as the most active. A 200 bp single-stranded oligodeoxynucleotide (ssODN) template was designed encoding the intended S2035T mutation and including an EcoR1 restriction site in a nearby intron for genotyping purposes. We mutated the sgRNA recognition and PAM sites of the ssODN by introducing several silent mutations to prevent subsequent sgRNA-directed cutting. The sequence of the ssODN is:

(SEQ ID NO: 1)
TCCCAATCGTAGCTTCCTGTGGTGTATCTGAATTCTTTCTCCCTTT
AATCCAGGTGAGTGAAGAGCTGATTCGGGTAGCCATCCTCTGGCAT
GAGATGTGGCATGAAGGATTGGAAGAAGCAACTCGCCTGTACTTCG
GAGAGAGGAACGTGAAAGGCATGTTTGAGGTGCTGGAGCCCCTGCA
TGCTATGATGGAACGG.

SgRNA and ssODN combinations were transfected into NIH/3T3 cells using Lipofectamine 3000 (Thermo Fisher Scientific) for initial validation purposes. The sgRNA (50 ng/ul), the ssODN containing the S2035T mutation (50 ng/ul), and recombinant SpCas9 protein (50 ng/ul) were mixed and microinjected by the UT Southwestern Transgenic Core into the cytoplasm of NOD/SCID one-cell fertilized eggs that were generated by in vitro fertilization.

Surviving eggs were transferred into the oviduct of pseudo-pregnant ICR recipient females. At the time of weaning, putative founder mice underwent tail snips to isolate DNA that was used for genotyping by PCR and EcoR1 restriction digestion. The founders were then confirmed by Sanger sequencing and WGS. WGS was performed by BGI Genomics, and data were processed by the Quantitative Biomedical Research Center at UT Southwestern Medical Center.

Fluorescence-activated cell sorting of TG and TME cells

Tissues were minced into small pieces and enzymatically digested for 10 minutes using 400 μg/mL collagenase P (Roche), 400 μg/mL dispase (Invitrogen) and 100 μg/mL DNase (Sigma) in RPMI-1640 (Sigma) containing 10% FBS (Sigma) at 37° C. Digested tissue was filtered using a 70 μm nylon filter (Falcon), and incompletely digested tissues were digested one more time. Single-cell suspensions were washed with 1% BSA (Research Products International) in PBS (Sigma). Cells were incubated with cell type-specific antibodies aSMA (1:100, Alexa Fluor 488-conjugated, #53-9760-82, eBioscience) and CAIX (1:100, Alexa Fluor 647-conjugated, #FAB2188R, R&D Systems) for 30 minutes. P-S6 was detected using an antibody against phospho-S6 Ser240/244 conjugated to PE (1:400, #14236, Cell Signaling Technology). We used True-Nuclear Transcription Factor Buffer Set (BioLegend) according to the manufacturer's instructions. After washing, the cells were analyzed by a FACSCalibur flow cytometer (BD Biosciences). FlowJo was used to analyze the data.

### Cell Culture and Transfection

Primary RCC cell lines were generated using established protocols (1). Primary RCC cell lines were cultured in high-glucose Dulbecco's Modified Eagle's Medium (DMEM) (Sigma), 10 ng/mL EGF (Life Technologies), 1% MEM nonessential amino acids (Gibco), 0.4 μg/mL hydrocortisone (Sigma), 1% penicillin/streptomycin (Gibco), and 10% FBS (Sigma).

Fibroblasts were derived from NOD/SCID^{mTOR-WT} and NOD/SCID^{mTOR-ST} Embryos. Briefly, embryos at 13.5 dpc were collected and minced into small pieces. Pieces were digested by 0.05% trypsin-EDTA (Sigma) for 15 minutes, followed by culturing in high-glucose DMEM (Sigma), 1% penicillin/streptomycin (Gibco), and 10% FBS (Sigma).

NIH/3T3 and HeLa cells were cultured in high-glucose DMEM (Sigma), 1% penicillin/streptomycin (Gibco), and 10% FBS (Sigma). All cells were maintained in a 37° C. incubator with 5% C02.

Plasmids [pCDH (#72299, Addgene) with mTOR-WT (lab plasmid database ID #958) or mTOR-ST (lab plasmid database ID #959)] were transfected into HeLa cells using Lipofectamine 2000 (Thermo Fisher). mTOR sgRNA and ssODN were transfected into NIH 3T3 cells using Lipofectamine 3000 (Thermo Fisher).

### Western Blot

Tumor (or cell) samples were suspended in ice-cold lysis buffer [25 mM Tris•HCl pH 7.4 (Research Products International), 150 mM NaCl (Sigma), 1% NP-40 (Fluka Biochemika), 1 mM EDTA (Research Products International), and 5% glycerol (Sigma)], supplemented with protease and phosphatase inhibitors [protease inhibitors: 0.1 μm aprotinin (USB Corporation), 0.02 mmol/L leupeptin (USB Corporation), 0.01 mmol/L pepstatin (USB Corporation), 0.5 mmol/L PMSF (Sigma); 2 mmol/L imidazole (Sigma), 1.15 mmol/L sodium molybdate (Sigma), 1 mmol/L sodium orthovanadate (Sigma), 5 nmol/L microcystin (Calbiochem)] and crushed with a pestle. Samples were transitioned to dry ice and homogenized until freezing began to occur. Samples were transitioned between wet ice and dry ice for ˜4 cycles until well homogenized and were further processed through QIAShredder spin columns (QIAGEN). Protein concentrations were measured using the Bio-Rad Protein Assay. Cell lysates were boiled in 3× loading buffer [10% SDS (Research Products International), 33% glycerol (Sigma), 300 mM DDT (Roche), 0.2% Bromophenol Blue (Sigma)], and western blotting was performed. Primary antibodies for phospho-S6 (Ser240/244) from Cell Signaling Technology (#5364S, 1:1000), total S6 from Bethyl Laboratories (#A300-556A, 1:1000), and α-Tubulin from Sigma Aldrich (#T5168, 1:5000) were used. Secondary antibodies were goat anti-rabbit IgG-HRP (#31460, 1:1000, Thermo Fisher) and goat anti-mouse IgG-HRP (#31430 1:2000, Thermo Fisher).

### Real-Time PCR

cDNA was synthesized using iScript™ Reverse Transcription Supermix for RT-qPCR (Bio-Rad). RT-qPCR was performed on a Bio-Rad CFX96 Real-Time PCR system using iTaq Universal SYBR Green Supermix (Bio-Rad). Primers were synthesized by Invitrogen. Primers available upon request.

### Immunohistochemistry and Immunofluorescence

All immunohistochemistry was performed using a DAKO® system and previously described protocols(1). p-S6 Ser240/244 (#5364S, Cell Signaling Technology) and Ki67 antibodies were applied at a 1:400 and 1:100 dilutions, respectively, with a secondary antibody from EnVision FLEX/HRP (#K800021, Agilent). The percentage of p-S6-positive cells in each tissue section was recorded by an investigator blinded to the treatment allocation. An H-score was derived from the product of the percentage of positive cells multiplied by intensity. The intensity of the staining was scored as: 0=no staining, 1=weak, 2=moderate, 3=strong (only 2 or 3 were considered positive). For Ki67 quantification, staining cells were quantified using the IHC profiler plugin in Image J. The quantification was performed on 20× images of 5 randomly selected high power fields (hpfs) per mouse and n=3 per condition.

For immunofluorescence, tumor tissues were fixed in 4% paraformaldehyde overnight, dehydrated in 30% sucrose (Research Products International) for 6 hours, embedded in OCT (Fisher Scientific), kept in a −80° C. freezer, and sectioned on a Leica cryostat in 10 μm slices. Cells were fixed with 4% paraformaldehyde (ChemCruz) for 15 minutes. Fixed tissue sections or cells were blocked with 0.3% Triton X-100 (Sigma) and 3% BSA (Research Products International) in PBS (Sigma) for 30 minutes at room temperature, incubated with primary antibody [to phospho-S6 (Ser240/244) (#5364S, 1:400), PAX8 (#10336-1-AP, 1:500, Proteintech), Vimentin (#ab8979, 1:500, Abcam)] at 4° C. overnight and detected by secondary antibodies with Alexa Fluor 488 goat-anti mouse (#A32723, 1:1000, Thermo Fisher) and Alexa Fluor 594 goat-anti rabbit (#A32740, 1:1000, Thermo Fisher). Nuclei were stained by DAPI (#13271, Thermo Fisher).

For co-staining experiments, formalin-fixed paraffin-embedded (FFPE) sections were subjected to an automated program of Leica Autostainer XL for dewaxing and rehydration. Subsequently tissue sections underwent antigen retrieval using EnVision™ FLEX Target retrieval solution (pH=9) at 90° C. for 20 min. Tissue sections were quenched in 0.1% glycine, permeabilized in 0.1% tritonX-100 and blocked in 5% BSA for 1 h. Sections were then incubated with an admixture of PAX8 antibody (#CI 438 A, 1:200 dilution, BioCare) (or a α-SMA antibody (#A2547, Sigma) at 1:200 dilution) and phospho-S6 (1:200 dilution) at 4° C. overnight. After washing with PBS, the sections were incubated with a mixture of anti-rabbit Alexa fluor 488 and anti-mouse Alexa fluor 584 secondary antibodies (1:200) at RT for 1 h. The staining was visualized and captured by Keyence microscopy. Quantification of single-positive (α-SMA or PAX8) or dual positive (α-SMA+P-S6+ or PAX8+P-S6+) cells was performed on 40× images of 5 randomly selected high power fields (hpfs) per section for each mouse (n=3) under each condition in a blind manner. Approximately, 800-1000 cells per mouse (n=3 per condition) were manually quantified using Image J.

### Co-Culture Assay

For co-cultures, primary tumor cells were plated at 1.5×105 cells/well in 6-well plates, and fibroblasts were plated at 1×105 in the insert of a 0.4 um transwell permeable support (Fisher Scientific). The next day, both primary cells and fibroblasts were washed once with PBS (Sigma). Transwell inserts were transferred to 6-well plates. 50 nM rapamycin or vehicle was used for treatment for 3 days.

### Reverse-Phase Protein Array (RPPA) Analysis

Tumor lysates were sent to MD Anderson Cancer Center for RPPA analysis were they were processed according to an established protocol (3, 4) (mdanderson.org/research/research-resources/core-facilities/functional-proteomics-rppa-core/rppa-process.html). Cellular proteins were denatured with 1% SDS (Research Products International) with beta-mercaptoethanol (Sigma) and diluted in five 2-fold serial dilutions in dilution buffer (lysis buffer containing 1% SDS). Serial-diluted lysates were arrayed on nitrocellulose-coated slides (Grace Biolab) using an Aushon 2470 Arrayer (Aushon BioSystems). A total of 5808 array spots were arranged on each slide, including spots corresponding to positive and negative controls prepared from mixed cell lysates or dilution buffer, respectively.

Each slide was probed with a validated primary antibody plus a biotin-conjugated secondary antibody. Only antibodies with a single or dominant band on western blotting and a Pearson correlation coefficient between RPPA and western blotting of greater than 0.7 were used. The signal obtained was amplified using a Dako Cytomation-catalyzed System and visualized by DAB colorimetric reaction. The slides were scanned, analyzed, and quantified using Microvigene customized software to generate spot intensity. Each dilution curve was fitted with a logistic model (“Supercurve Fitting”; bioinformatics.mdanderson.org/OOMPA). This fits a single curve using all of the samples (i.e., dilution series) on a slide, with the signal intensity being the response variable and the dilution steps being the independent variable. The fitted curve is plotted with the signal intensities—both observed and fitted—on the y-axis and the log 2 concentration of proteins on the x-axis. Protein concentrations of each set of slides are then normalized by median polish, which is corrected across samples by the linear expression values using the median expression levels of all antibody experiments to calculate a loading correction factor for each sample.

### Statistics

Bar charts and series plots are presented as the mean±standard error of the mean (SEM) from more than three independent experiments. Unless otherwise indicated, the statistical significance was determined by t-test using GraphPad Prism. For each XP line, ANOVA with post-hoc t-tests were used to test for differential (phospho)protein levels between the three arms in the RPPA analysis (vehicle-treated, rapamycin-sensitive, and rapamycin-resistant) with FDR-adjusted P-values. For the mixed model analysis, data were summarized as mean tumor growth per day with 95% confidence intervals, as well as by fold change between groups. A positive fold change indicates increased tumor growth rates in the first group compared to the second. Estimates were obtained from a three-way factorial mixed model analysis for treatment group, mouse type, and day of measurement. Baseline volume was also included as a term in the model to control for differences in starting volumes. An autoregressive (AR-1) covariance structure was used to account for the correlation among repeated measurements for each mouse. ANOVA and mixed model analyses were completed using SAS 9.4. A P-value <0.05 was considered significant.

### Example 2: Rapamycin Inhibits mTORC1 in ccRCC TGs and Suppresses Growth

This example covers modeling rapalog resistance in mice to understand how resistance to rapalogs develops. TGs from three ccRCC patients (XP144, XP164, and XP490) were implanted subcutaneously into mice. Once they reached a suitable size (˜300 mm³), mice were treated with either rapamycin or vehicle. Rapamycin (also called sirolimus) accounts for 70% of the circulating drug following temsirolimus administration in patients. Rapamycin was administered to mice in a regimen matching human exposures (0.5 mg/kg every 48 hours). FIG. 1A provides growth curves of subcutaneously implanted TGs (XP144, XP164 and XP490) in NOD/SCID mice treated with vehicle (Ve) or rapamycin (Ra). The error bars indicate standard error of the mean (SEM; n>3 per condition). As shown in this figure, rapamycin effectively suppressed TG growth.

FIG. 1B provides representative phospho-S6 immunohistochemistry images of TGs from vehicle-treated (Ve) mice or mice treated with rapamycin collected while TGs were sensitive (˜day 28) (Ra^{sensitive}) and after development of resistance (Ra^{Resistant}). FIG. 1C displays quantitated results from FIG. 1B, and includes error bars that indicate SEM (n=3 per condition). FIG. 1D provides Western blot analyses of mTORC1 pathway status of TGs from vehicle-treated mice or mice treated with rapamycin collected while TGs were sensitive (Ra^{sensitive}) and after development of resistance (Ra^{Resistant}). For each condition, 3 different TGs are shown per TG line. P-S6, phospho-S6 Ser240/244.

As shown in these figures, rapamycin inhibition was accompanied by suppression of mTORC1, as demonstrated by western blot and immunohistochemical (IHC) analyses at day 28 (FIG. 1B-D; Ra^{sensitive}). All three TG lines, which were initially sensitive to rapamycin, acquired resistance with prolonged treatment (FIG. 1A). Resistance developed with variable latency (FIG. 1A). This feature was reproducible across the three TG lines, indicating that it was intrinsically linked to the particular tumor. Unexpectedly, despite unequivocal growth of TGs after acquisition of resistance, mTORC1 remained inhibited (FIG. 1B-D; Ra^{Resistant}). This was observed by both IHC and western blot. Indeed, the level of suppression of S6 phosphorylation in resistant TGs was indistinguishable from sensitive TGs harvested at day 28.

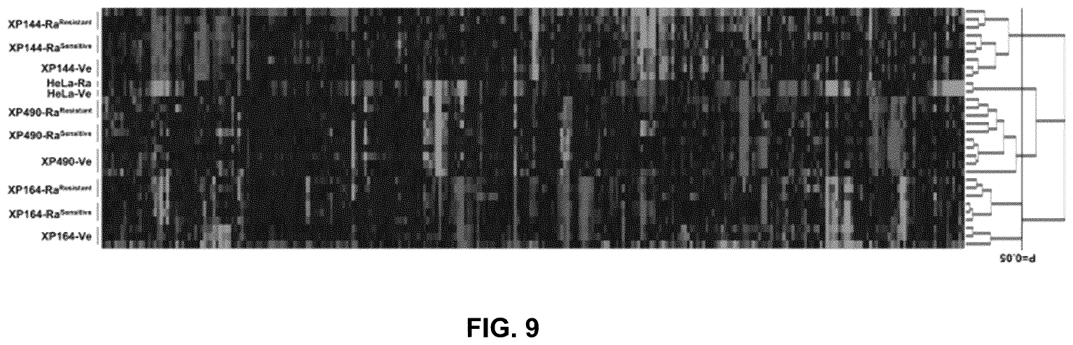

Reverse-phase protein array analysis (RPPA) was performed for 279 (phospho)proteins. Results from these analyses are summarized in FIG. 1E, which provides RPPA analyses of (phospho)protein levels of rapamycin-sensitive and rapamycin-resistant TGs compared with vehicle-treated tumors for each TG line. ANOVA with post-hoc t-tests was used to test for differential (phospho)protein levels between the three arms for each TG line, with FDR-adjusted p-values. The figure provides a heatmap of selected (phospho)protein levels of TGs from vehicle-treated mice or mice treated with rapamycin collected while TGs were sensitive (Ra^{sensitive}) and after development of resistance (Ra^{Resistant}). Unsupervised hierarchical clustering was used to group TGs by the most similar protein expression profiles. TGs clustered according to their respective source, indicating that expression profiles were more dependent on the parent tumors from which the TGs were derived than on rapamycin treatment (FIG. 9). Of the 279 (phospho)proteins probed, the only two with shared alterations among all TG groups were phospho-S6 (p-S6) Ser 235/236 and Ser 240/244 (all P<0.05). Consistent with western blot and IHC assays, p-S6 was suppressed not only in rapamycin-sensitive (day 28) but also rapamycin-resistant tumors (FIG. 1E). While there were some proteins with statistically significant changes between responsive and resistant tumors, none other was consistent across the three TGs (Table 1), which summarizes RPPA analyses of (phospho)protein levels of rapamycin-sensitive and rapamycin-resistant TGs compared with vehicle-treated tumors for each TG line. ANOVA with post-hoc t-tests were used to test for differential (phospho)protein levels between the three arms for each TG line, with FDR-adjusted P-values. T: Significant upregulation compared to Vehicle; 1: Significant downregulation compared to Vehicle;−: No significant change compared to Vehicle. It was observed, however, that proteins implicated in the cell cycle increased after the development of resistance, which is consistent with resumption of cell proliferation compared to day 28. While it might have been expected other mTORC1 targets to be similarly deregulated as S6, S6 is a particularly robust marker, and failure to reactivate S6 phosphorylation upon acquisition of resistance unequivocally shows persistent mTORC1 inhibition.

TABLE 1
Proteomic Analyses of Rapamycin Impact in Sensitive and Resistant Tumorgrafts.

| | | FDR-adjusted P | | | |
|---|---|---|---|---|---|
| | | | Sensitive | Resistant | Sensitive |
| | | Overall | vs. | vs. | vs. |
| Sensitive | Resistant | ANOVA | Vehicle | Vehicle | Resistant |

XP 144

| | | | | | | |
|---|---|---|---|---|---|---|
| IRF-1 | ↑ | ↑↑ | 0.0118 | 0.0090 | 0.0013 | 0.0130 |
| Raptor | ↑ | ↑ | 0.0118 | 0.0054 | 0.0017 | |
| S6_pS235_S236 | ↓ | ↓ | 0.0064 | 0.0003 | 0.0021 | |
| S6_pS240_S244 | ↓ | ↓ | 0.0064 | <0.0001 | 0.0021 | |

XP 164

| | | | | | | |
|---|---|---|---|---|---|---|
| Annexin-I | — | ↑ | 0.0013 | | <0.0001 | 0.0012 |
| Chk2 | ↓ | — | 0.0322 | 0.0048 | | 0.0075 |
| Cyclin-D1 | ↓ | — | 0.0350 | 0.0048 | | 0.0075 |
| FAK_pY397 | ↓ | — | 0.0050 | 0.0022 | | 0.0029 |
| GPBB | — | ↑ | 0.0109 | | 0.0048 | 0.0008 |
| MEK1 | ↓ | ↑ | 0.0229 | 0.0156 | 0.0227 | 0.0054 |
| MIF | — | ↑ | 0.0229 | | 0.0076 | 0.0039 |
| S6_pS235_S236 | ↓ | ↓ | 0.0050 | 0.0048 | 0.0007 | |
| S6_pS240_S244 | ↓ | ↓ | 0.0050 | 0.0048 | 0.0006 | |
| SHP-2_pY542 | ↓ | — | 0.0275 | 0.0075 | | 0.0440 |
| p21 | ↓ | — | 0.0229 | 0.0048 | | 0.0066 |

XP 490

| | | | | | | |
|---|---|---|---|---|---|---|
| Aurora-B | — | ↑ | 0.0454 | | 0.0083 | 0.0133 |
| CDC2-p34 | ↓ | — | 0.0454 | 0.0016 | | 0.0145 |
| CDK1 | ↓ | ↑ | 0.0176 | 0.0063 | 0.0142 | 0.0076 |
| Cyclin-B1 | ↓ | ↑ | 0.0365 | 0.0227 | 0.0164 | 0.0083 |
| FoxM1 | ↓ | — | 0.0454 | 0.0016 | | 0.0480 |
| MDM2_pS166 | ↓ | — | 0.0454 | 0.0045 | | 0.0112 |
| PLK1 | ↓ | ↑ | 0.0492 | 0.0335 | 0.0302 | 0.0124 |
| Pdcd4 | ↑ | ↑ | 0.0365 | 0.0231 | 0.0003 | |
| Rb_pS807_S811 | ↓ | ↑ | 0.0013 | 0.0018 | 0.0137 | 0.0007 |
| S6_pS235_S236 | ↓ | ↓ | 0.0007 | 0.0005 | 0.0006 | |
| S6_pS240_S244 | ↓ | ↓ | <0.0001 | 0.0002 | 0.0002 | |
| Smad3 | ↓ | — | 0.0454 | 0.0045 | | 0.0197 |

### Example 3: Rapamycin Resistance is Lost Upon Subsequent Transplantation and in Cells Culture

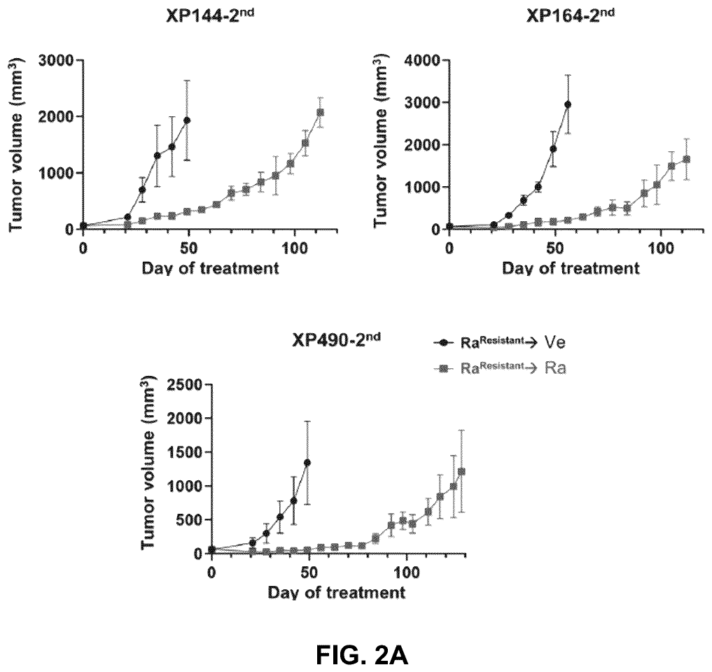

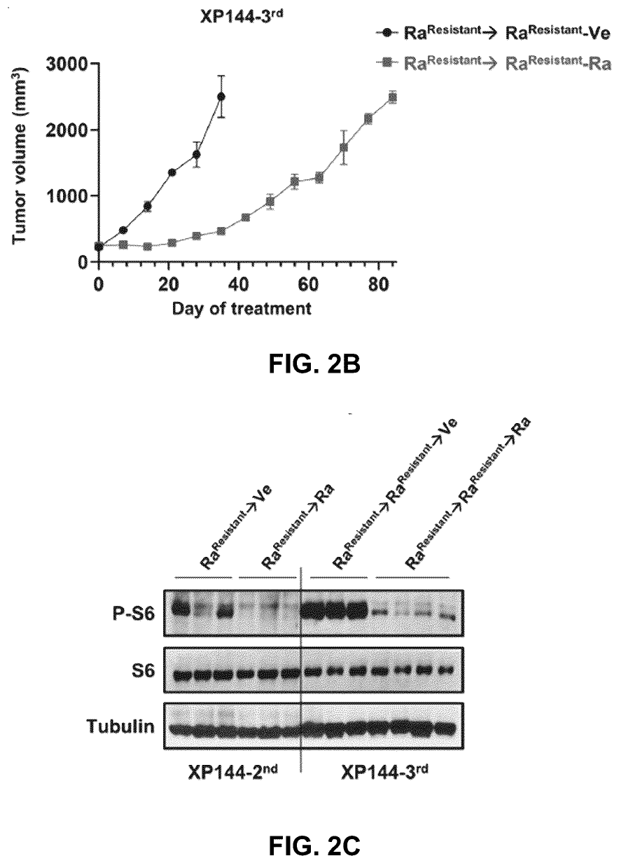

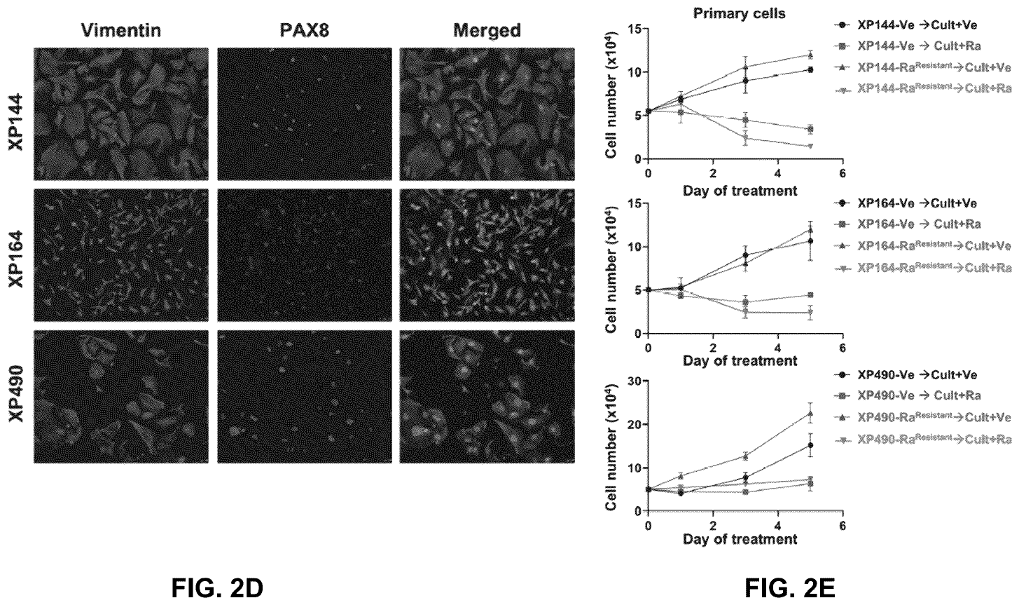

A further investigation was performed to determine whether resistance would be maintained upon passage to a subsequent cohort of mice. The results of these analyses are summarized in FIGS. 2A-C. FIG. 2A provides TG growth curves following transplantation of rapamycin-resistant tumors to a new cohort of mice. The error bars indicate SEM (n>_3 per condition). FIG. 2B is a representative TG growth curve of rapamycin-resistant tumor transplanted into a third mouse cohort. The error bars indicate SEM (n>_3 per condition). FIG. 20 provides results from representative western blot analyses of sequentially transplanted tumors in second and third cohorts (n>_3 per condition).

TGs that had acquired resistance were thus passaged to a second cohort of mice. Interestingly, rapamycin was able to inhibit the growth of these previously resistant TGs (FIG. 2A). Furthermore, while resistance could again be acquired after prolonged treatment, the latency periods were similar to those in the first mouse cohort (FIG. 2A). Similar results were observed when resistant tumors were passaged to a third cohort of mice (FIG. 2B). Western blot analyses showed similar results as in the first mouse cohort with persistent mTORC1 suppression despite reacquisition of resistance (FIG. 2C).

It was next investigated whether primary cell lines derived from resistant TGs would remain resistant in vitro. Primary cells were generated from both vehicle-treated and rapamycin-resistant TGs. The primary cells were then analyzed with vimentin and PAX8 immunofluorescence imaging. The results of these analyses are shown in FIG. 2D, which is a series of immunofluorescent staining images of TG-harvested primary cells with RCC markers, vimentin and PAX8. In this figure, vimentin stains are shown in the leftmost column, PAX8 stains are shown in the middle column, and an overlay of the vimentin and PAX8 stains are shown in the rightmost column.

After validation, primary tumor cell lines were treated with vehicle or rapamycin. Primary cell growth curves from vehicle-treated or Ra^{Resistant} TGs in mice cultured with vehicle (Ve) or 50 nM rapamycin (Ra) are shown in FIG. 2E. The error bars indicate SEM (n>3 per condition). It was found that primary cell lines derived from resistant TGs were sensitive to rapamycin in culture indicating that rapamycin resistance was lost.

Together these data show that rapamycin resistance is lost when tumors are explanted and evaluated either in vitro or in subsequent mouse cohorts. These data demonstrate that resistance is transient and can be lost, which is most consistent with a non-genetic mechanism.

In keeping with this notion, sequencing analyses failed to identify MTOR mutations in resistant TGs. This was not surprising, in particular since mTORC1 remained inhibited in TGs despite resistance acquisition (FIGS. 1B-E).

### Example 4: Rapamycin Resistance is Accompanied by mTORC1 Reactivation in the Tumor Microenvironment

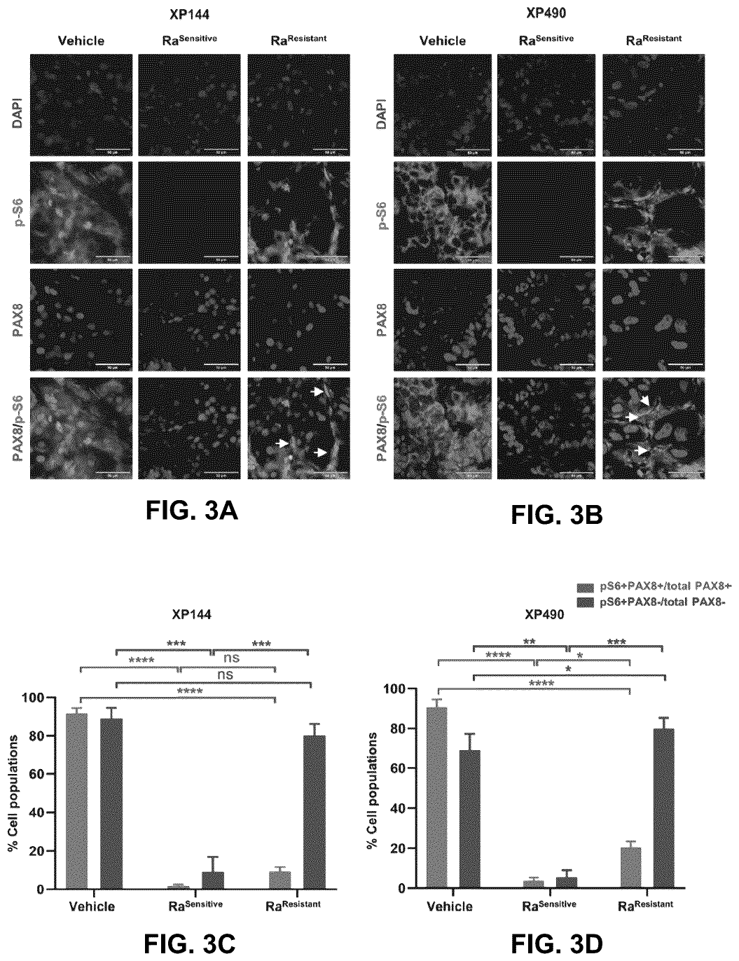

Dual immunofluorescence (IF) studies were performed to dissect the effects of rapamycin on the TGs. A PAX8 stain was utilized to distinguish tumor from non-tumor/stromal cells. A p-S6 stain was used to assess mTORC1 activity. Images of these attains are shown in FIG. 3A (XP144) and FIG. 3B (XP490). In these figures, the rows correspond to DAPI, p-S6, PAX8, and p-S6/PAX8 overlays, respectively from top to bottom. The columns correspond to vehicle treated, Ra^{sensitive}, and RA^{Resistant} mice. White arrows indicate pS6-stained stromal cells (PAX8−) in Ra^{Resistant} TGs. Consistent with previous data, p-S6 remained suppressed in PAX8− positive RCC cells even after the acquisition of resistance. In contrast, analyses of PAX8-negative cells (tumor microenvironment cells) showed p-S6 inhibition in sensitive tumors, but reactivation upon the acquisition of resistance (FIGS. 3A-D).

### Example 5: Generation of a Rapamycin-Resistant NOD/SCID Recipient Mouse to Test the Role of the TME

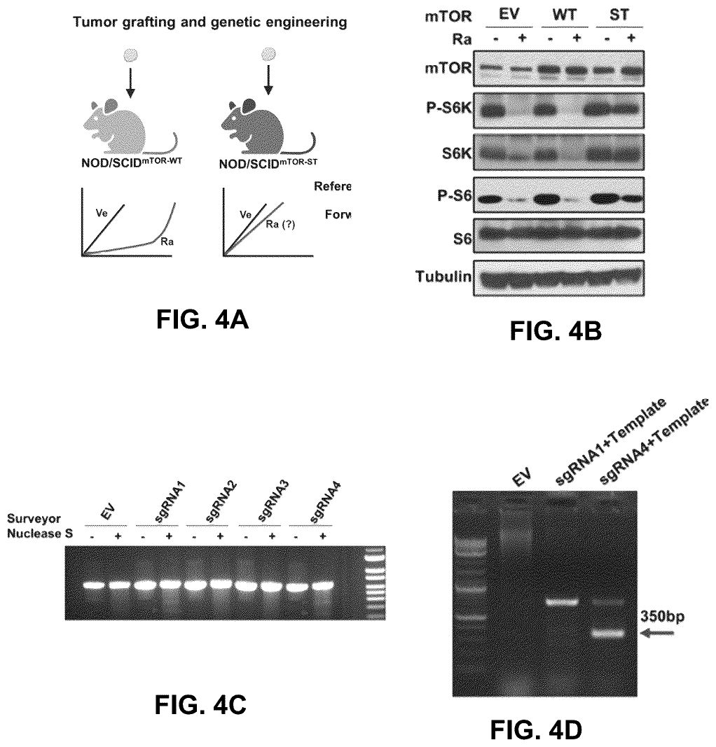

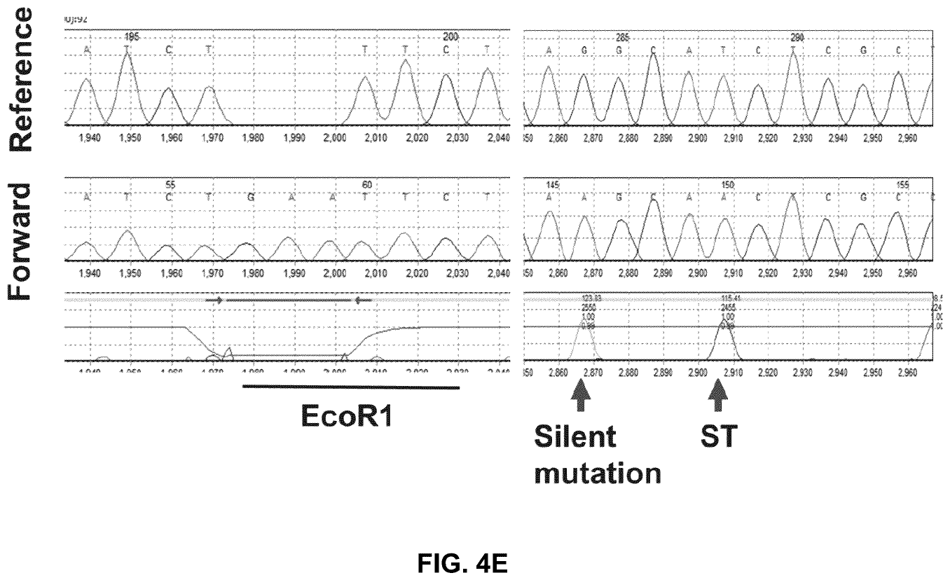

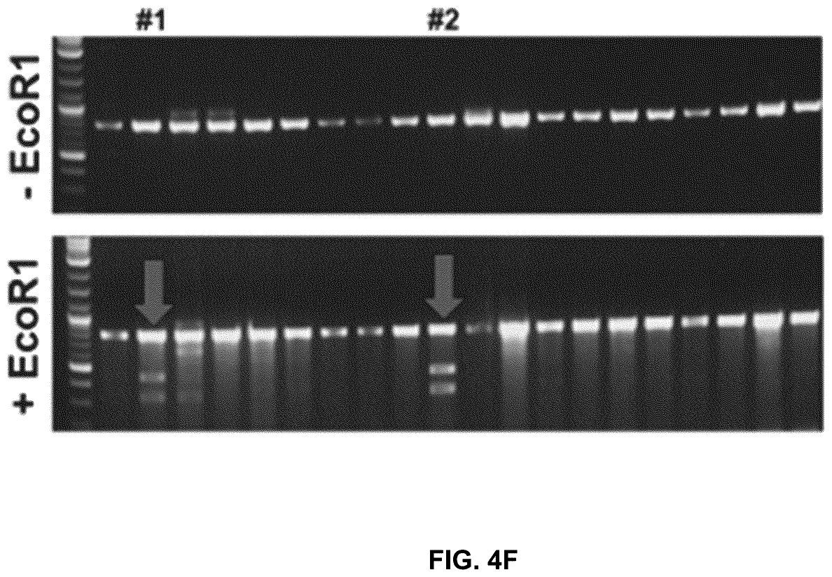

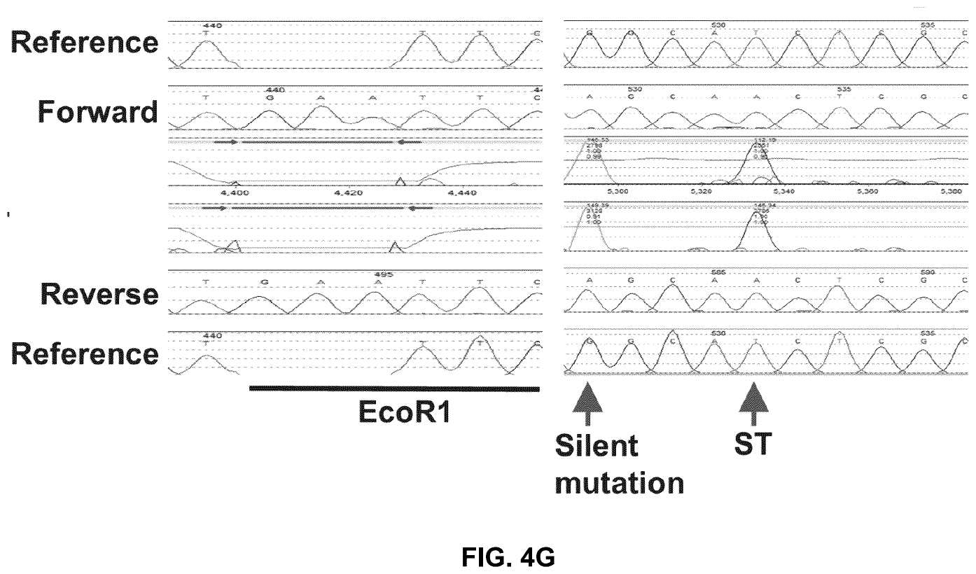

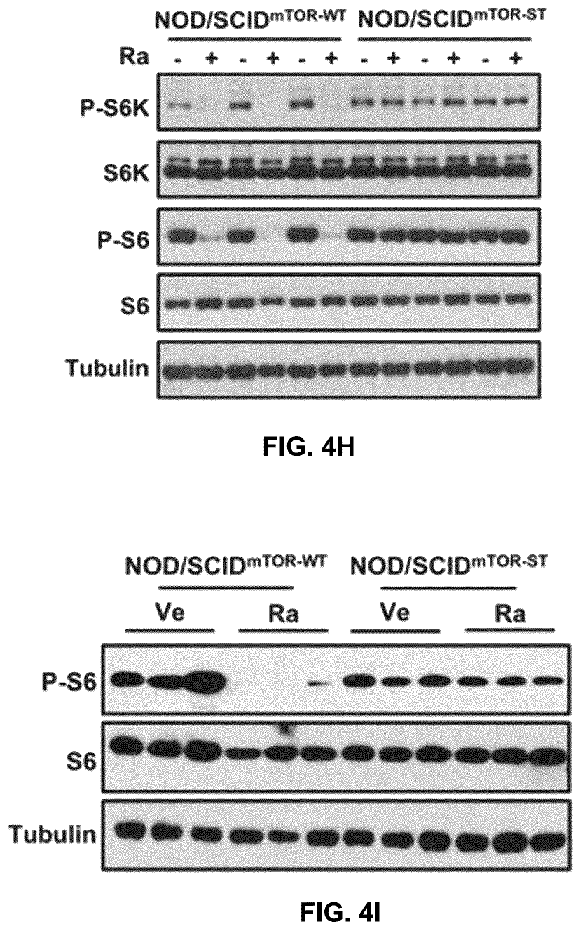

To determine what role the TME may play an important role in the development of resistance to rapamycin a model with constitutively active mTORC1 in the TME was generated. Two classical but distinct approaches to cancer modeling were integrated for these experiments tumor grafting and genetic engineering. This model is illustrated in FIG. 4A, which is a cartoon illustrating this mouse model mouse with combined tumorgraft (TG/PDX) and genetic engineering approaches to introduce a mutation in mTOR that confers rapamycin resistance to NOD/SCID host mice. Graphs represent TG growth over time in vehicle (Ve) and rapamycin-treated (Ra) wild-type and mutant mice, assuming a role of non-tumor cells in mediating rapamycin anti-tumor effects. Immunocompromised NOD/SCID recipient mice with an mTOR rapamycin resistance mutation were then generated. Rapamycin binds to the FRB domain of mTOR and mutation of serine 2035 to threonine (Ser^{1972/1975} in yeast TOR1/2) has been previously shown to confer resistance.

To evaluate this further, it was tested whether mTOR-ST (referring to mTOR S2035T) was sufficient to block mTORC1 inhibition by rapamycin in cultured cells. FIG. 4B provides results from Western blot analyses of HeLa cells transfected with empty vector (EV), wild-type mTOR (mTOR-WT) and mTOR S2035T (mTOR-ST) expression vectors and treated (or not) with rapamycin (50 nM, 45 min). As predicted, ectopic expression of mTOR-ST blocked the effects of rapamycin on S6 phosphorylation.

A strategy was developed to generate NOD/SCID mice with the mTOR-ST mutation using CRISPR/Cas9. Using the MIT CRISPR design tool, which is optimized to minimize off-target effects, 4 sgRNAs were designed and evaluated initially in mouse NIH/3T3 cells using the Surveyor assay. FIG. 4C provides DNA electrophoresis of Surveyor test results from sgRNAs targeting MTOR (or an empty vector (EV) control) in NIH/3T3 cells. Arrows indicate Surveyor nuclease S digestion fragments. Two sgRNAs were selected and designed the corresponding single-stranded oligodeoxynucleotide (ssODN) templates encoding the S2035T mutation. An EcoRI restriction site was introduced in a nearby intron for genotyping purposes and silent mutations in the PAM site to prevent re-cutting.

FIG. 4D provides a gel stain following selective DNA amplification of NIH/3T3 cells transfected with sgRNAs+template using a primer containing the EcoR1 consensus sequence in the ssODN template. FIG. 4E summarizes results from Sanger sequencing, and illustrates ssODN template utilization with mutation incorporation in NIH/3T3 cells. Pairwise combinations of the sgRNAs and ssODN templates were introduced into NIH/3T3 cells, and recombination was evaluated by PCR and Sanger sequencing.

FIG. 4F provides results from genotypic analyses of DNA from pups generated from the injection of sgRNAs (sgRNA1 and sgRNA4), ssODN (containing the S2035T mutation), and recombinant SpCas9 into fertilized nuclei of NOD/SCID mouse zygotes, by restriction fragment length polymorphism following PCR amplification and EcoR1 digestion for the introduced restriction site. Two positive founders were obtained (#1 and #2). Arrows indicate EcoRI digestion fragments that show the presence of the mutation. The most promising sgRNA and ssODN combination was then injected into NOD/SCID zygotes along with recombinant spCas9 protein. Two heterozygous founders were obtained (NOD/SCID^{mTOR-ST})

FIG. 4G provides results from bidirectional Sanger sequencing from founder mouse DNA, confirming introduction of resistance mutation, silent mutations, and the EcoRI site. The presence of the MTOR mutation was confirmed by Sanger sequencing. Whole-genome sequencing (WGS) of a founder mouse confirmed the intended MTOR mutation as summarized in Table 2, which provides mutation analyses from NOD/SCID^{mTOR-ST} founder compared to wild-type NOD-SCID mouse.

TABLE 2
MTOR Gene Mutation Analysis of Founder Mice By Whole-Genome Sequencing

| Position | Reference | Substitution | Gene | Region | Mutation | Transcript | Protein | Other |
|---|---|---|---|---|---|---|---|---|
| 148538706 | T | TGAA | MTOR | intron | | | | EcoR1 |
| | | | | | | | | site |
| 148538784 | C | A | MTOR | CDS | silent | c.6090C > A | | |
| 148538785 | C | T | MTOR | CDS | silent | c.6091C > T | | |
| 148538793 | G | A | MTOR | CDS | silent | c.6099G > A | | |
| 148538797 | T | A | MTOR | CDS | missense | c.6103T > A | p.Ser2035Thr | |
| 148538803 | T | C | MTOR | CDS | silent | c.6109T > C | | |
| 148538811 | T | C | MTOR | CDS | silent | c.6117T > C | | |
| 148538814 | G | A | MTOR | CDS | silent | c.6120G > A | | |

As summarized in Table 3, whole genome sequencing failed to identify other mutations that could be attributed to off-target sgRNA effects by comparison to wild-type NOD/SCID mice. Off-target regions predicted by Cas-OFFinder were queried from WGS of NOD/SCID^{mTOR-ST}founder mouse. No mutations were found in up to 1,000 base pairs (bps) flanking potential off-target sites

TABLE 3
Evaluation of Predicted Off-Target sgRNA
Regions By Whole-Genome Sequencing

| | Flanking window length (0-1,000 base | | | | |
|---|---|---|---|---|---|
| | pairs away from the predicted regions) | | | | |
| Region | 0 | 10 | 100 | 1,000 | |
| 1: 143334383-143334405 | 0 | 0 | 0 | 0 | off-target |
| 1: 175832802-175832824 | 0 | 0 | 0 | 0 | off-target |
| 2: 122177411-122177433 | 0 | 0 | 0 | 0 | off-target |
| 2: 142893175-142893197 | 0 | 0 | 0 | 0 | off-target |
| 4: 140369427-140369449 | 0 | 0 | 0 | 0 | off-target |
| 5: 138762498-138762520 | 0 | 0 | 0 | 0 | off-target |
| 6: 32008188-32008210 | 0 | 0 | 0 | 0 | off-target |
| 6: 88964030-88964052 | 0 | 0 | 0 | 0 | off-target |
| 9: 49307920-49307942 | 0 | 0 | 0 | 0 | off-target |
| 10: 30400140-30400162 | 0 | 0 | 0 | 0 | off-target |
| 11: 120492000-120492022 | 0 | 0 | 0 | 0 | off-target |
| 17: 19585460-19585482 | 0 | 0 | 0 | 0 | off-target |
| 19: 53574338-53574360 | 0 | 0 | 0 | 0 | off-target |

To test the impact of the mTOR-ST mutation, a founder mouse was bred with wild-type NOD/SCID mice, and generated mouse embryo fibroblasts. Fibroblasts from mutant (NOD/SCID^{mTOR-ST}) and wild-type littermates were treated with rapamycin and evaluated by Western blot. FIG. 4H shows Western blot results of MEFs derived from wild-type (n=3) or NOD/SCID^{mTOR-ST} mutant mice (n=3) treated (or not) with rapamycin (Ra) (50 nM, 45 min). Rapamycin failed to inhibit p-S6 in NOD/SCID^{mTOR-ST} fibroblasts indicating that even in the heterozygous state, mTOR-ST conferred rapamycin resistance.

These experiments were expanded by evaluating the impact of rapamycin on the kidneys of mutant mice. FIG. 41 provides Western blot analyses of mouse kidneys from either wild-type (n=3) or NOD/SCID^{mTOR-ST} mutant (n=3) 5-week-old mice treated with rapamycin (Ra; 0.5 mg/kg every 48 hours×5 treatments) or vehicle (Ve) and collected 2 hours after the last dose. By comparison to control mice, rapamycin failed to inhibit mTORC1 in the kidneys of 5-week-old NOD/SCIDmTOR-ST mice.

### Example 6: mTORC1 Inhibition in the Host is Necessary for Rapamycin Anti-Tumor Activity

XP144, XP164 and XP490 were then implanted into NOD/SCID^{mTOR-ST} mice. The tumors were given time to grow to about 300 mm³, at which time the mice were treated with rapamycin. If the observed reactivation of mTORC1 in the TME of resistant TGs contributed to resistance, it was hypothesized that constitutive mTORC1 activation in NOD/SCID^{mTOR-ST} mice may dampen rapamycin inhibition.

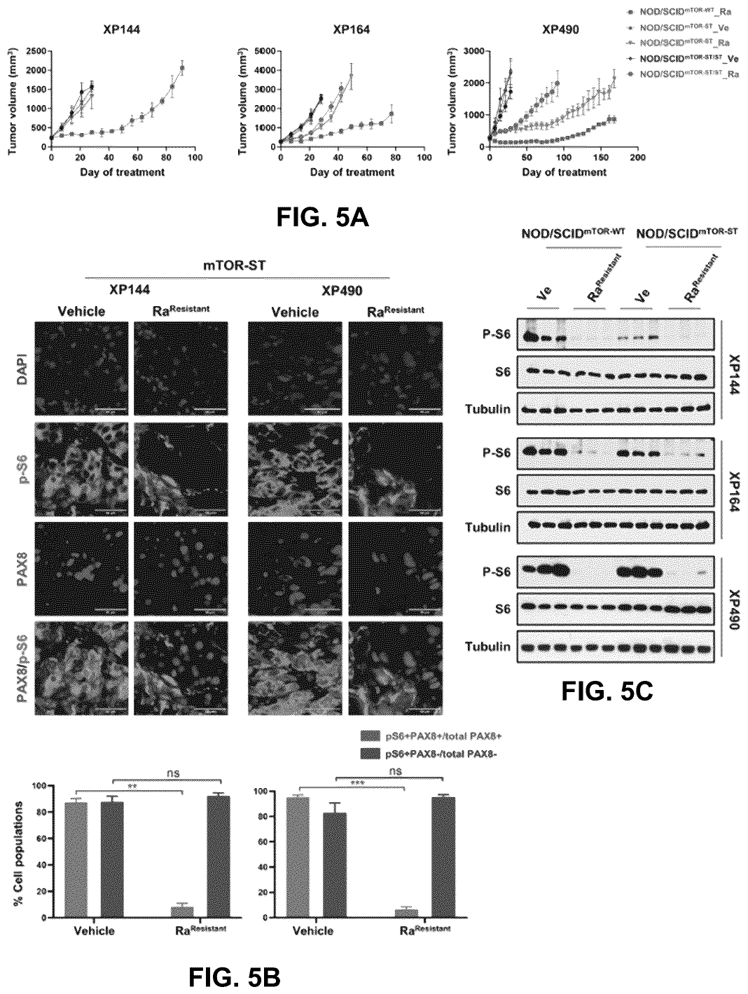

FIG. 5A summarizes the results of TG growth curves in wild-type (NOD/SCID^{mTOR-WT}) and mTOR S2035T heterozygous NOD/SCID^{mTOR-ST} (as well as homozygous NOD/SCID^{mTOR-ST/ST}) mice treated with rapamycin (Ra) or vehicle (Ve). The error bars indicate SEM (n>3 per condition). The ability of rapamycin to suppress TG growth in NOD/SCID^{mTOR-ST} mice was significantly impaired across all 3 TG lines. While the impact varied, the mTOR mutation consistently interfered with rapamycin-mediated tumor growth inhibition in the 3 different TG lines (FIG. 5A). The effect was most pronounced in XP144, where the mTOR-ST mutation completely suppressed the effects of rapamycin.

Linear mixed model analysis was used to quantitate the impact of the mTOR-ST mutation on tumor growth inhibition by rapamycin. In XP144, rapamycin reduced tumor growth by 1.5-fold in wild-type mice (P=0.036) but had no significant effect in NOD/SCID^{mTOR-ST} mice (P=0.34) (FIG. 5A and Table 4). A similar phenomenon was observed in XP164, where rapamycin delayed tumor growth in wild-type mice by 3.7-fold (P<0.0001) but had no significant effect in NOD/SCID^{mTOR-ST}mice (P=0.15). The mTOR-ST mutation had the least effect on XP490. In XP490, rapamycin reduced tumor growth by 11-fold in wild-type mice (P<0.0001), and the resistance mutation dampened the inhibition to 6-fold (P<0.0001). Interestingly, however, by making the mTOR-ST mutation homozygous in NOD/SCID mice, resistance was substantially increased (Table 4). By comparison to the 11-fold downregulation in tumor growth in wild-type mice, rapamycin inhibited tumor growth by 2-fold in mTOR-ST homozygous mice (P<0.0001). The observation that the introduction of an mTOR resistance mutation in recipient mice significantly dampened the anti-tumor effects of rapamycin showed that mTORC1 inhibition in non-tumor cells is necessary for rapamycin anti-tumor effect. In Table 4, data are summarized with mean tumor growth rates per day (95% confidence intervals) with fold change between the two groups. A positive fold change indicates increased tumor growth rates in the first group. mTOR-WT, wild type mice; mTORST, heterozygous mTOR-ST mice; mTORST/ST, homozygous mTOR-ST mice. Rapa, rapamycin-treatment; Veh, vehicle-treatment

TABLE 4
Mixed-Model ANOVA of Rapamycin Impact
on Tumor Growth in Different Hosts

| | | |
|---|---|---|
| Mean tumor growth | Fold | |
| per day (95% CI) | change | P |

XP144

| | | | |
|---|---|---|---|
| mTOR^{WT}-Rapa vs. | 20.4 (10.5, 30.3) vs. | −1.5 | 0.036 |
| mTOR^{WT}-Veh | 51.9 (24.2, 79.6) | | |
| mTOR^{ST}-Rapa vs. | 54.6 (36.8, 72.4) vs. | −0.3 | 0.34 |
| mTOR^{ST}-Veh | 68.5 (45.5, 91.4) | | |
| XP164 | | | |
| mTOR^{WT}-Rapa vs. | 18.3 (12.6, 24.0) vs. | −3.7 | <0.0001 |
| mTOR^{WT}-Veh | 86.8 (66.5, 107.2) | | |
| mTOR^{ST}-Rapa vs. | 67.6 (57.2, 78.0) vs. | −0.3 | 0.15 |
| mTOR^{ST}-Veh | 89.5 (61.7, 117.3) | | |
| mTOR^{ST/ST}-Rapa vs. | 69.5 (58.5, 80.6) vs. | −0.3 | 0.13 |
| mTOR^{ST/ST}-Veh | 92.7 (64.9, 120.5) | | |

XP490

| | | | |
|---|---|---|---|
| mTOR^{WT}-Rapa vs. | 5.1 (1.1, 9.1) vs. | −10.7 | <0.0001 |
| mTOR^{WT}-Veh | 59.6 (47.5, 71.7) | | |
| mTOR^{ST}-Rapa vs. | 15.0 (11.7, 18.3) vs. | −5.8 | <0.0001 |
| mTOR^{ST}-Veh | 102.7 (87.4, 117.9) | | |
| mTOR^{ST/ST}-Rapa vs. | 23.8 (18.8, 28.8) vs. | −2.1 | <0.0001 |
| mTOR^{ST/ST}-Veh | 73.6 (53.6, 93.5) | | |

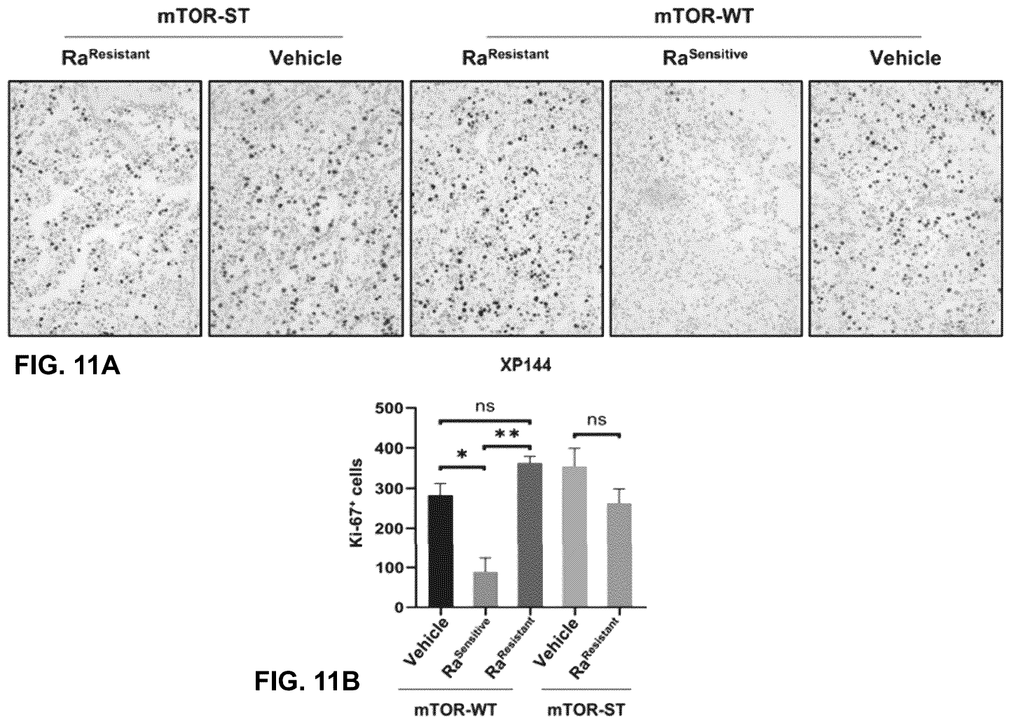

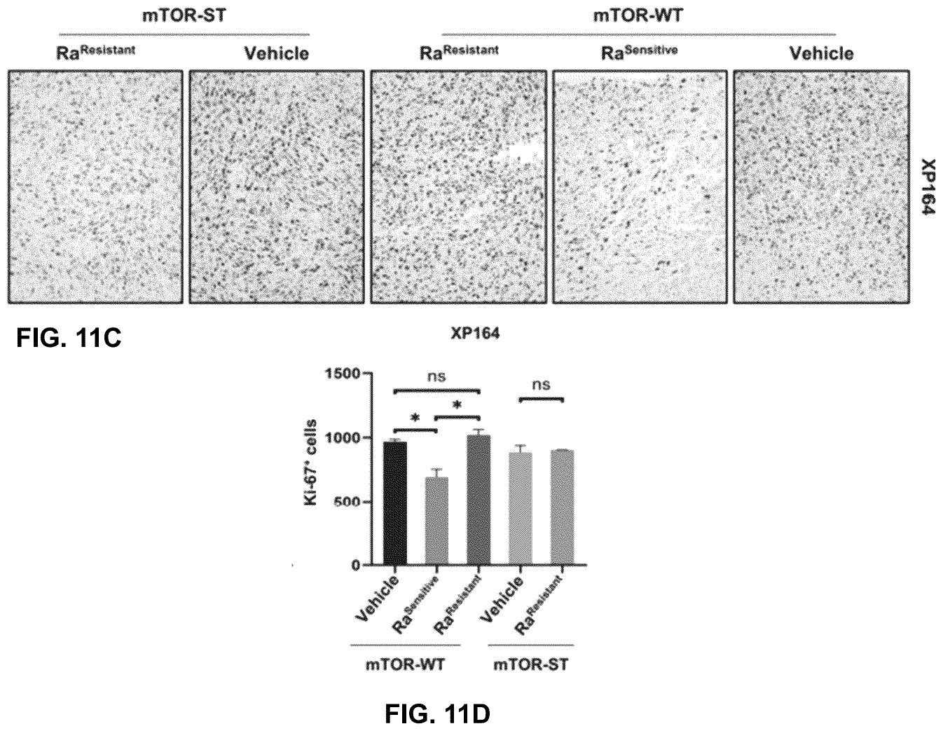

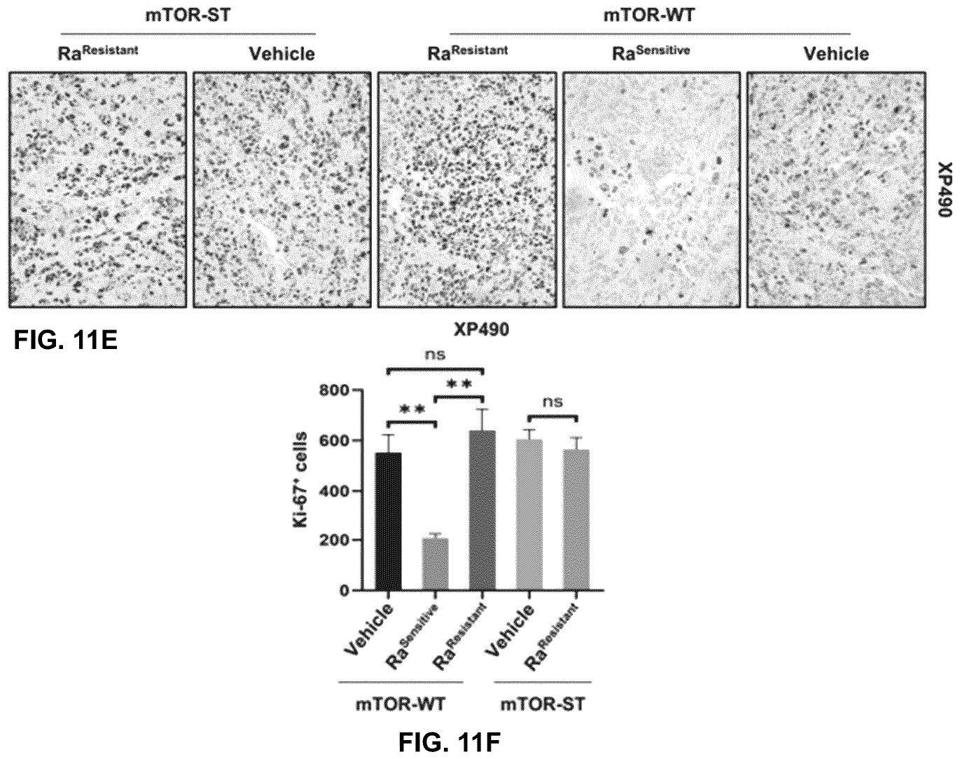

To characterize the phenomenon further, cell proliferation analyses were performed by staining the tissues of TGs with Ki67. FIGS. 11A, C, and E show Ki67 staining in 3 TGs, XP144, XP164 and XP490. Ki67+cells were quantified by assessing stained cells using the IHC profiler plugin in Image J. FIGS. 11B, D and E display the quantification performed on images of 5 randomly selected high power fields (hpfs) per mouse and n=3 per condition. The data indicated that the rapamycin downregulated TG cell proliferation, but cell proliferation was increased after the development of resistance and was maintained in mTOR-ST mice, despite treatment with rapamycin.

### Example 7: Constitutive mTORC1 Activation in Non-Tumor Cells Induces Resistance to Rapamycin Despite mTORC1 Suppression in the Tumor

Two scenarios were conceived to explain mTORC1 inhibition. One where constitutive mTORC1 activation in non-tumor cells somehow interfered with mTORC1 suppression by rapamycin in the tumor. In the second scenario, mTORC1 would be activated in the TME, but still inhibited in tumor cells. This later scenario would be consistent with the acquired resistance model, where mTORC1 remained inhibited in tumor cells, but not in the TME (FIG. 3). IHC and western blot analyses were performed to test which scenario was possible.

The results of these analyses are shown in FIGS. 5B-C. FIG. 5B provides results from immunofluorescence analyses (PAX8 and p-S6) of TGs in mTOR S2035T mice (XP144, NOD/SCID^{mTOR-ST}; XP490, NOD/SCID^{mTOR-ST/ST}) treated with rapamycin (Ra) or vehicle (Ve). Scale bars are 50 μm (top). Bar graph represents percent cell populations with p-S6 staining in PAX8+ (tumor) and PAX8− (stromal) cells in vehicle/rapamycin treatment groups in an mTOR-ST background. The error bars indicate SEM; **, p<0.001; ***, p<0.0001; ns, not significant. FIG. 5C displays results from Western blot analyses of XP144, XP164 and XP490 TG in wild-type and NOD/SCID^{mTOR-ST} mice treated with vehicle of after acquisition of resistance to rapamycin (n=3 per condition). p-S6 and S6 were from gels run in parallel with the same extracts. P-S6, phospho-S6 Ser240/244.

As shown in these figures, rapamycin profoundly inhibited mTORC1 in tumor cells even in the presence of the mTOR-ST mutation. Furthermore, even by western blot, there were little differences in the magnitude of mTORC1 inhibition of TG in wild-type and mTOR-ST mice (FIG. 5C). Thus, these data show that TG growth was restored despite persistent mTORC1 inhibition in tumor cells. These data are consistent with the notion that resistance conferred by the host is not mediated by mTORC1 reactivation in tumor cells. To further evaluate this notion, dual IF studies were performed on TGs in mTOR-ST mice. While mTORC1 activity was suppressed by rapamycin in PAX8-positive cancer cells, mTORC1 was active in PAX8-negative host-derived mutant stromal cells (FIG. 5B).

A key component of the TME is cancer-associated fibroblasts (CAFs). There are several types of CAFs, including myofibroblasts, which are characterized by expression of α-smooth muscle actin (α-SMA). Accordingly, dual IF staining was performed with antibodies against α-SMA and p-S6.

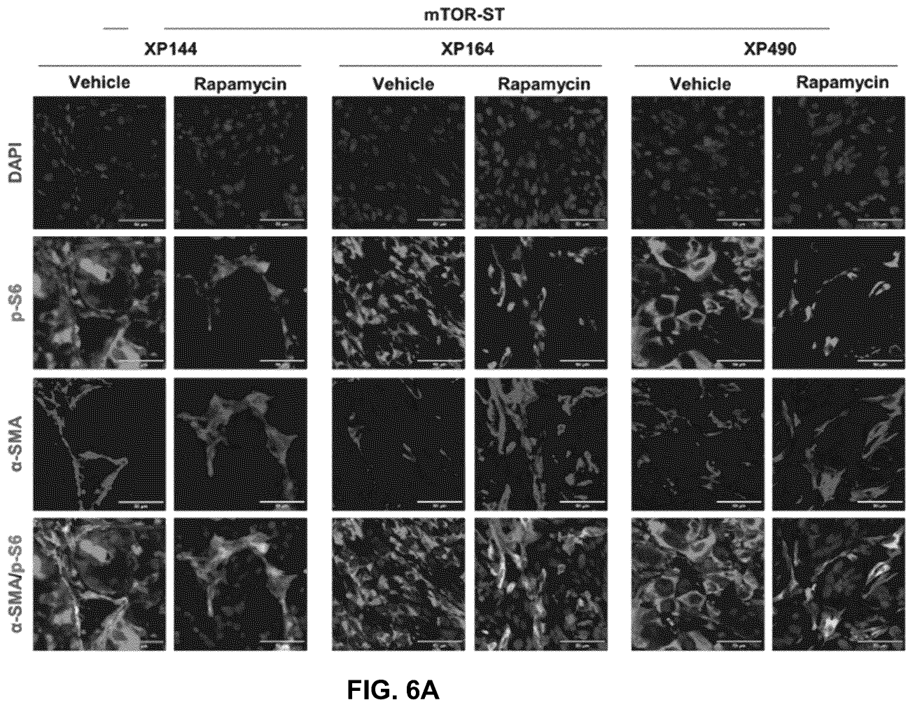

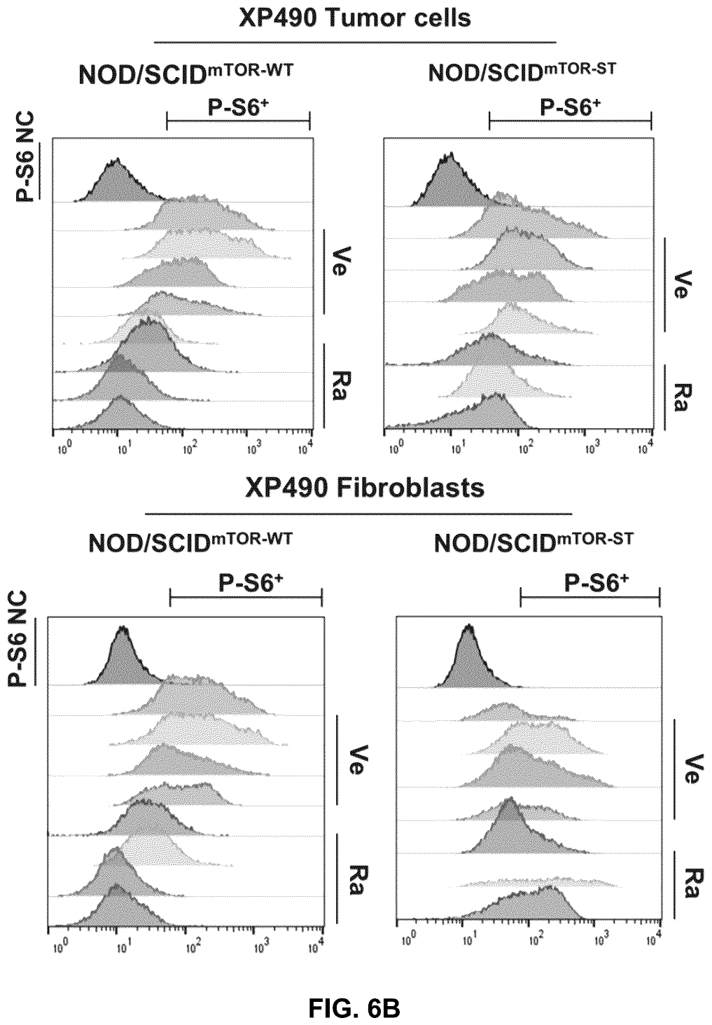

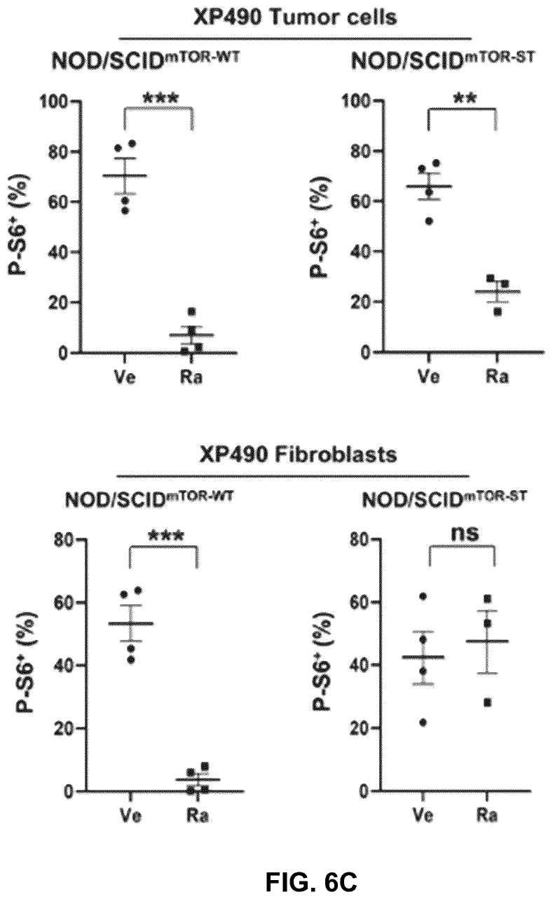

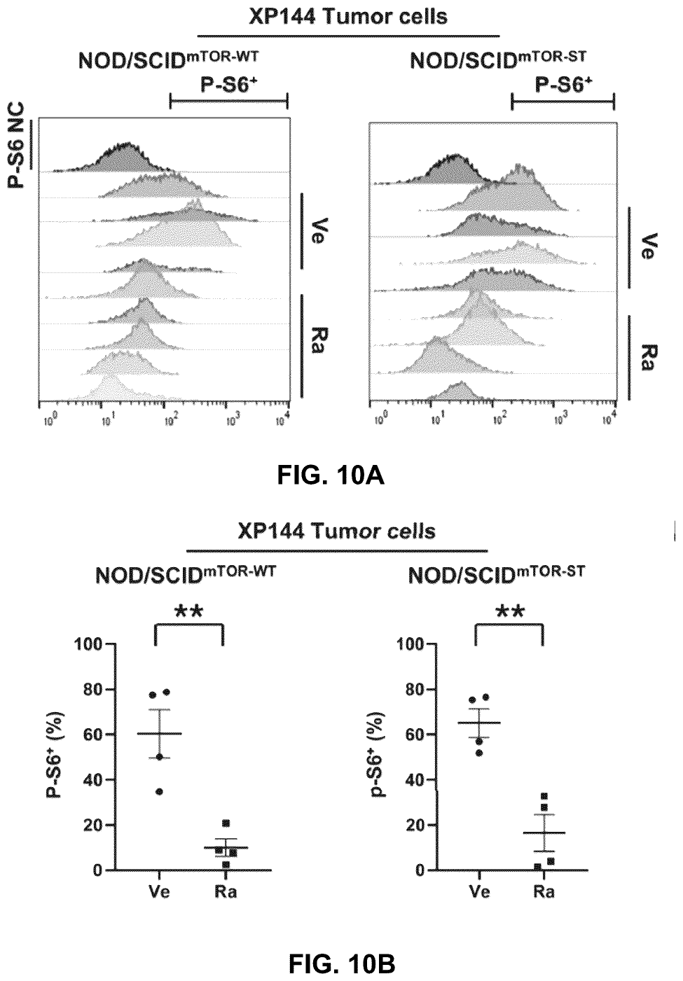

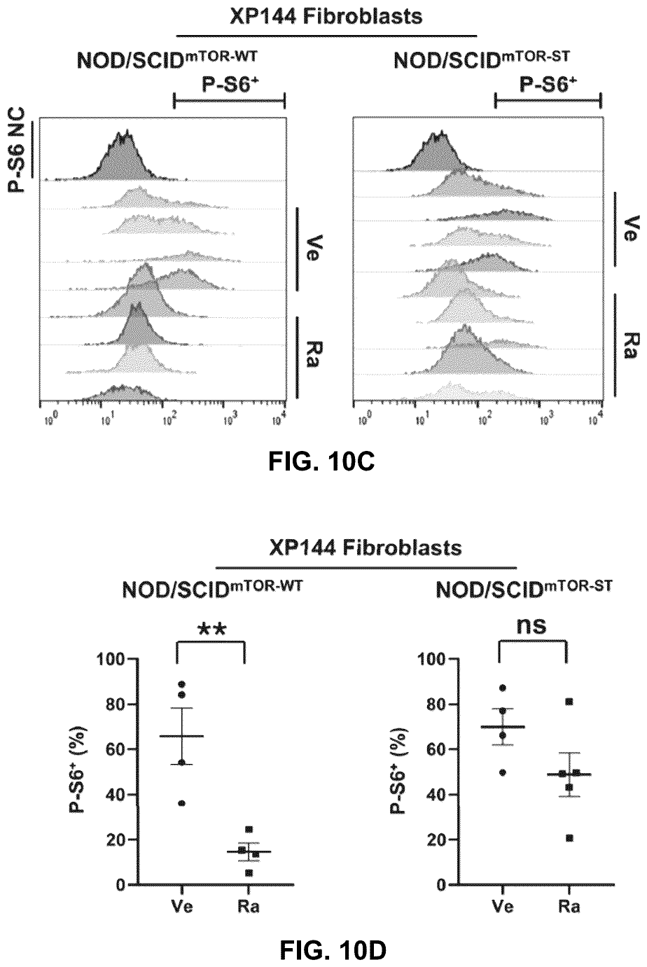

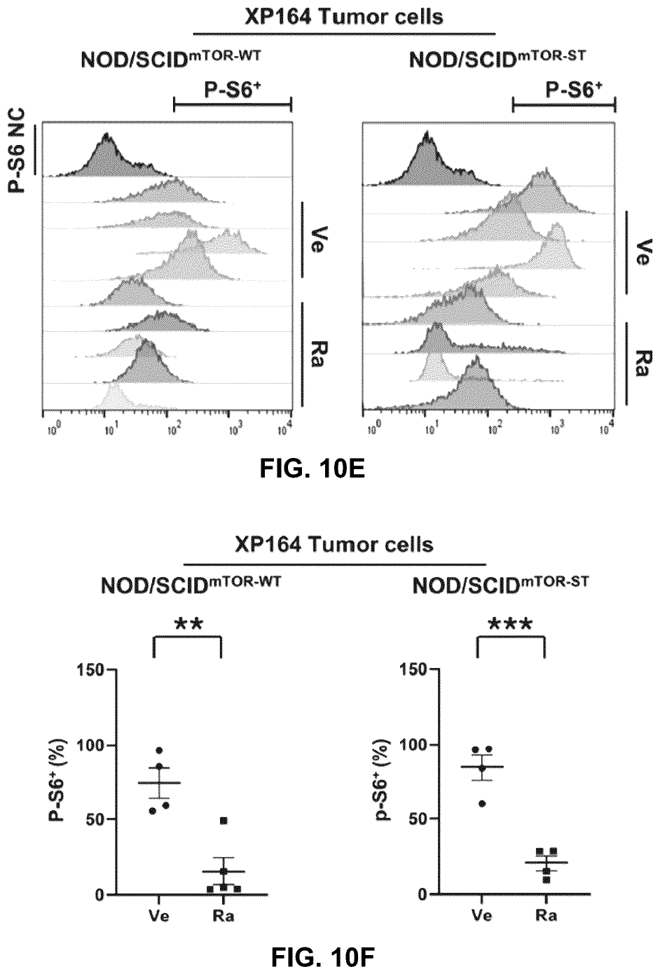

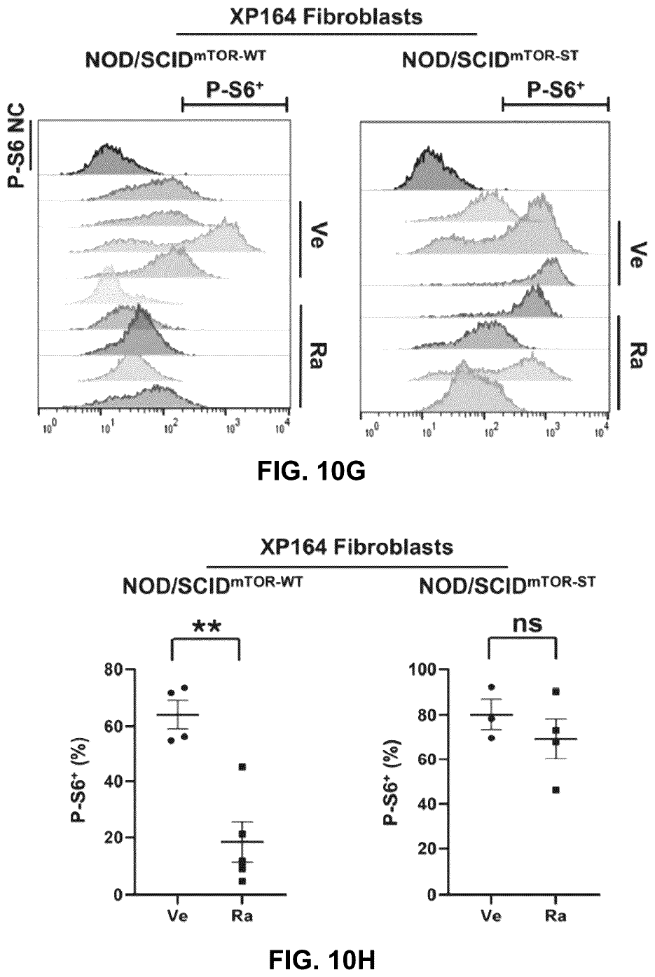

The results of these analyses are provided in FIGS. 6A-C. FIG. 6A summarizes results from immunofluorescence analyses (α-SMA [fibroblasts] and p-S6) of XP144 TGs (NOD/SCIDmTOR-ST) as well as XP164 and XP490 TGs (NOD/SCIDmTOR-ST/ST) mice treated with rapamycin (Ra) or vehicle (Ve). FIG. 6B and FIG. 10 summarize the distribution of p-S6 signal by FACS in tumor cells (CAIX-positive) and myofibroblasts (α-SMA-positive) from TGs implanted into wild-type and NOD/SCID^{mTOR-ST} mice and treated with vehicle or rapamycin compared to a negative control (NC, without phospho-S6 antibody). P-S6, phospho-S6 Ser240/244. FIG. 6C provides quantitation of the results shown in FIG. 6B and FIG. 10 and includes error bars that indicate SEM; **, p<0.01; ***, p<0.001; ns, not significant.

These immunofluorescence studies showed that mTOR-ST mutant myofibroblasts retained mTORC1 activity despite rapamycin treatment. Fluorescence-activated cell sorting (FACS) experiments were performed to expand these results. TGs were disaggregated and subjected to FACS to analyze tumor cells (identified using the CAIX ccRCC cell surface marker) and myofibroblasts (using anti-SMA antibodies). While the p-S6 distribution in tumor cells shifted leftwards by rapamycin treatment, it was largely unaffected in myofibroblasts^{mTOR-ST} (FIGS. 6B, 6C and 10). Thus, TG resistance to rapamycin in NOD/SCID^{mTOR-ST} mice is associated with inhibition of mTORC1 in tumor cells, but persistent mTORC1 activation in myofibroblasts.

### Example 8: Resistant TGs Explanted from NOD/SCID^{mTOR-ST} Become Sensitive When Transplanted into NOD/SCID Wild-Type Mice

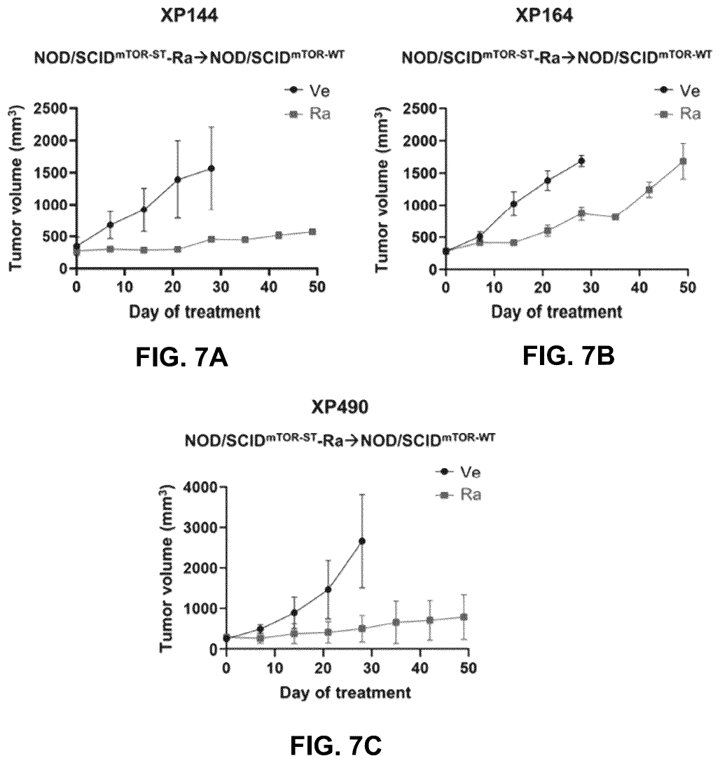

To further assess the role of the TME, rapamycin-resistant tumors from NOD/SCID^{mTOR-ST} mice were transplanted into NOD/SCID^{mTOR-WT} mice. The results of these analyses are shown in FIGS. 7A-7C, which are graphs of tumorgraft (TG) growth curves of rapamycin-resistant tumors transplanted from NOD/SCID^{mTOR-ST} mice into a new cohort of wild-type mice and treated with vehicle (Ve) or rapamycin (Ra). The error bars indicate SEM (n>3 per condition). It was found that resistant tumors became sensitive to rapamycin in wild-type mice. Thus, resistance was lost when transplanting tumors back into sensitive hosts, which is in keeping with an essential role for mTORC1 in non-tumor cells.

### Example 9: Inhibition of Tumor Cells by Rapamycin is Dampened by NOD/SCID^{mTOR-ST} Fibroblasts in Co-Culture Experiments

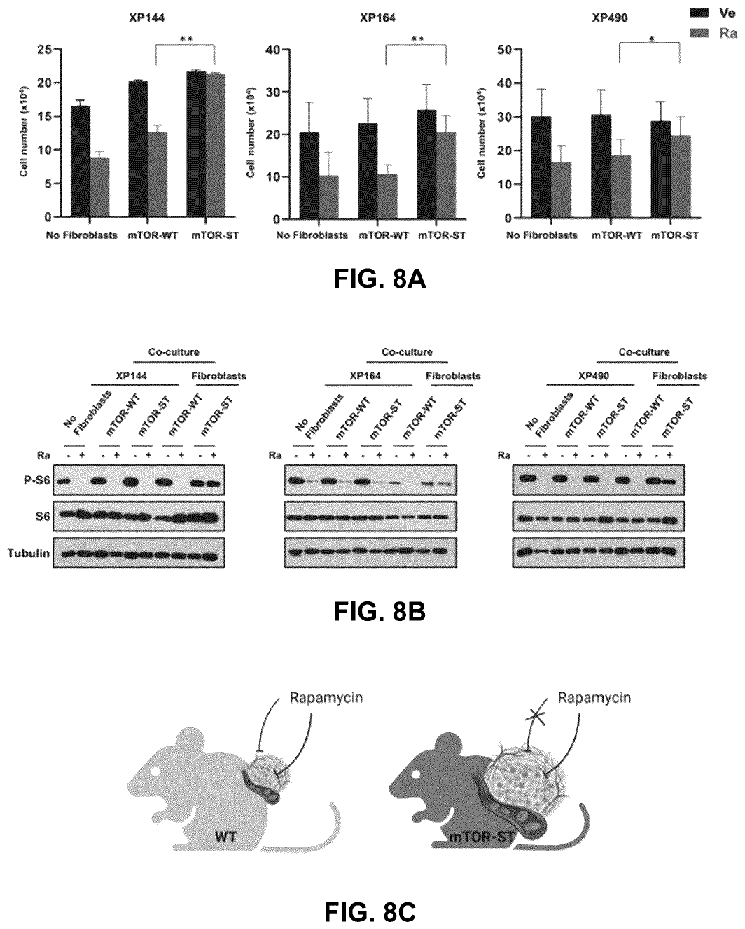

Primary cells from each TG line were co-cultured with mouse embryo fibroblasts (either wild-type or mTOR-ST mutant) and the impact on cell proliferation was evaluated by counting cells. The results of these analyses are shown in FIGS. 8A-B. FIG. 8A summarizes quantitation experiments of primary cells from XP144, XP164 and XP490 TGs co-cultured with wild-type or NOD/SCID^{mTOR-ST} fibroblasts and treated with vehicle or 50 nM rapamycin for 3 days. The error bars in this figure indicate SEM (n=3 per condition; *, p<0.05; using a paired t-test with co-cultures from the same experiment). FIG. 8B summarizes results from Western blot analyses of the co-cultured primary tumor cells or fibroblasts treated with vehicle or rapamycin. While wild-type fibroblasts did not affect tumor cell inhibition by rapamycin, this was significantly dampened by the mutant fibroblasts (FIG. 8A). In keeping with the findings in mice, western blotting showed that p-S6 was suppressed by rapamycin in tumor cells but not in mutant fibroblasts (FIG. 8B).

Thus, experiments in vitro using fibroblasts recapitulate the findings in vivo, where constitutive activation of mTORC1 in non-tumor cells is sufficient to support tumor cell proliferation despite the lack of mTORC1 activity. Furthermore, inasmuch as the ability of fibroblasts in culture to support tumor cell proliferation is contingent upon themselves being resistant to rapamycin, the data show that the signal provided by the fibroblasts is mTORC1-dependent. However, as mTORC1 remained inhibited in the tumor cells, this signal does not appear to act by restoring mTORC1 activity in tumor cells, but rather by enabling tumor cells with suppressed mTORC1 to proliferate. This model of rapalog action and resistance is illustrated in FIG. 8C. As depicted in this figure, failure to inhibit mTORC1 in the tumor microenvironment of NOD/SCID^{mTOR-ST} mice induces resistance, enabling tumor growth despite persistent mTORC1 inhibition in tumor cells.

Although the disclosure has been described with reference to the presently preferred embodiment, it should be understood that various modifications can be made without departing from the spirit of the disclosure.

1-63. (canceled)

64. A method of identifying a subject for treatment with an mTOR1 inhibitor, the method comprising:
a) i) contacting the mTORC1 inhibitor to a cell derived from a tumor microenvironment of the subject or mTORC1 isolated from the cell and detecting inhibition of mTORC1 in the cell or the mTORC1 isolated from the cell; and/or ii) analyzing a cell derived from a tumor microenvironment from the subject for an mTORC1 inhibitor resistance marker; and
b) identifying the subject for treatment with the mTORC1 inhibitor if mTORC1 inhibition is detected and/or if the mTORC1 inhibitor resistance marker is not detected.

65. The method of claim 64, wherein detecting mTORC1 inhibition comprises measuring a level of phosphorylation of the mTORC1, S6 kinase, ribosomal S6, or 4EBP, or a combination thereof.

66. The method of claim 64, wherein the method comprises detecting inhibition of mTORC1 isolated from the cell.

67. The method of claim 64, wherein step a) i) comprises:
1) measuring a decrease in ATP hydrolysis by the mTORC1 isolated from the cell; and/or
2) measuring a level of binding of the mTORC1 inhibitor to the mTORC1 isolated from the cell or a level of binding of the mTORC1 inhibitor to FK506-binding protein 12 (FKBP12) isolated from the cell; and/or
3) precipitating the mTORC1 isolated from the cell and measuring activity of the mTORC1 in the presence of a protein substrate and ATP.

68. The method of claim 67, wherein the protein substrate is S6 kinase or 4EBP.

69. The method of claim 64, wherein analyzing the cell for an mTORC1 inhibitor resistance marker comprises profiling the subject for a genetic mutation.

70. The method of claim 69, wherein the genetic mutation is associated with increased or constitutive mTORC1 activity.

71. The method of claim 64, wherein the cell derived from a tumor microenvironment of the subject is an immune cell, stromal cell, or fibroblast.

72. The method of claim 64, the method further comprising:
d) repeating detecting inhibition of mTORC1 in the cell or the mTORC1 isolated from the cell and/or repeating analyzing a cell derived from a tumor microenvironment from the subject for an absence of the mTORC1 inhibitor resistance marker; and
e) continuing treatment with the mTORC1 inhibitor if mTORC1 inhibition is detected and/or the mTORC1 inhibitor resistance marker is absent.

73. The method of claim 72, wherein d) is performed about 3 days to 600 days, about 3 days to 15 days, about 10 days to 50 days, about 20 days to 100 days, about 40 days to 200 days, about 60 days to 300 days, or about 120 days to 600 days after c).

74. The method of claim 64, wherein the detecting is in vitro.

75. The method of claim 64, wherein the cancer is renal cancer.

76. The method of claim 75, wherein the renal cancer is clear-cell renal cell carcinoma or non-clear-cell renal cell carcinoma.

77. The method of claim 64, wherein the mTORC1 inhibitor is rapamycin, MTI-31, dactolisib, deforolimus, ridaforolimus, temsirolimus, everolimus, zotarolimus, RAD001, CCI-779, KU-0063794, WYE-354, GSK1059615, AZD8055, nitazoxanide, trokinib, palomid 529, PP121, OSI-027, NU7441, omipalisib, WYE-125132, WYE-687, WAY-600, sapanisertib, torin 1, torin 2, zotarolimus, onatasertib, CZ415, PZR620, or an analogue thereof.

78. The method of claim 77, wherein the mTORC1 inhibitor is rapamycin, deforolimus, ridaforolimus, temsirolimus, everolimus, zotarolimus, RAD001, CCI-779, or an analogue thereof.

79. A method of treating a subject with a cancer comprising:
a) administering an mTORC1 inhibitor to the subject for a first time period;
b) ceasing administration of the mTORC1 inhibitor for a second time period following the first time period if the disease has been determined to be resistant to the mTORC1 inhibitor; and
c) resuming administration of the mTORC1 inhibitor following the second time period for a third time period.

80. The method of claim 79, wherein the cancer has been determined to be resistant to the mTORC1 inhibitor when a cell derived from the tumor microenvironment from the subject is resistant to mTORC1 inhibition and/or has an mTORC1 inhibitor resistance marker.

81. The method of claim 80, further comprising measuring the resistance to the mTORC1 inhibitor and/or detecting an mTORC1 inhibitor resistance marker in a cell derived from the tumor microenvironment from the subject about every 1, 2, 3, 4, 5, 6, 7, 8, 9, 10, 12, 15, 20, 25, 30, 40, 50, or 60 days.

82. The method of claim 79, wherein the second time period is about 2 to 60 days, about 2 to 5 days, about 2 to 10 days, about 2 to 20 days, about 5 to 20 days, about 5 to 40 days, about 10 to 40 days, about 10 to 60 days, about 20 to 60 days, about 30 to 60 days, or about 40 to 60 days.

83. A method of treating a subject with an mTORC1 inhibitor, the method comprising administering the mTORC1 inhibitor to the subject, wherein the subject has been identified for treatment with the mTORC1 inhibitor according to a method of claim 64.

84. A method of determining the role of the tumor microenvironment in response to a therapy comprising:
i) transplanting a first tumor or tumor cell into a first immunocompromised mammal comprising a mutation and a second immunocompromised mammal that does not comprise the mutation;
ii) administering the therapy to the first and second immunocompromised mammals; and
iii) comparing a response of the tumor or tumor cell in the first and second immunocompromised mammals; thereby determining the role of the tumor microenvironment in response to the therapy, wherein the mutation is an mTOR1 mutation and the therapy comprises an mTOR1 inhibitor; the mutation is an EGFR (epidermal growth factor receptor) mutation and the therapy comprises an EGFR inhibitor; the mutation is an Ab kinase mutation and the therapy comprises an Abl kinase inhibitor; or the mutation is a Ret mutation (i.e., a RET proto-oncogene mutation) and the therapy comprises a Ret inhibitor.
